(19) United States
(12) Patent Application Publication
(10) Pub. No.: US 2026/0009803 A1
(43) Pub. Date: Jan. 8, 2026

(54) ORGAN AGING BIOMARKERS DERIVED FROM THE PLASMA PROTEOME

(71) Applicant: The Board of Trustees of the Leland Stanford Junior University, Stanford, CA (US)

(72) Inventors: Jarod Rutledge, Stanford, CA (US); Hamilton Oh, Stanford, CA (US); Benoit Lehallier, Stanford, CA (US); Anton Wyss-Coray, Stanford, CA (US)

(21) Appl. No.: 18/993,670

(22) PCT Filed: Jul. 17, 2023
(86) PCT No.: PCT/US2023/027896

§ 371 Date: Jan. 13, 2025

Related U.S. Application Data
(60) Provisional application No. 63/389,689, filed on Jul. 15, 2022.

(51) Int. Cl. G01N 33/68; G16B 40/00; G16H 10/40; G16H 50/30

(52) U.S. Cl. CPC G01N 33/6893; G16B 40/00; G16H 10/40; G16H 50/30; G01N 2800/7042

(57) ABSTRACT

Provided herein are minimally invasive compositions, methods, systems, kits and uses for biomarkers derived from the plasma proteome that identify, predict, and monitor organ health, aging, dysfunction and disease in humans. Said methods comprise a) obtaining a sample from said subject; b) measuring the concentrations of two or more proteins from said organ in said sample from said subject wherein said concentrations of said two or more proteins provides a biological age of said organ in health and/or disease; and c) comparing said biological age of said organ to a chronological age of said subject, wherein a gap between said biological age of said organ and said chronological age of said subject identifies accelerated and slowed aging of said organ.

### CROSS-REFERENCE TO RELATED APPLICATIONS

The present Application claims priority to U.S. Provisional Application Ser. No. 63/389,689 filed Jul. 15, 2022, the entirety of which is incorporated by reference herein.

### FEDERAL FUNDING

This invention was made with Government support under contracts AG047366, AG066515, and AG072255 awarded by the National Institutes of Health. The Government has certain rights in the invention.

### FIELD

Provided herein are minimally invasive compositions, methods, systems, kits and uses for biomarkers derived from the plasma proteome that identify, predict, and monitor organ health, aging, dysfunction and disease in humans.

### BACKGROUND

Evidence in animal models indicates that aging is heterogeneous not only between individuals but also within an individual, and that organs age at different rates. However, assessing differences in organ aging in living persons is an unresolved challenge at present. Unique compositions of the blood plasma proteome are needed to identify organ-specific assays of aging in living organisms including humans.

### SUMMARY

Provided herein are minimally invasive compositions, methods, systems, kits and uses for biomarkers derived from the plasma proteome that identify, predict, and monitor organ health, aging, dysfunction and disease in humans.

In some embodiments, the present invention provides unique aging signatures for 15 organs in the plasma proteome, and machine learning panels that reproducibly predict organ age in 3 independent human cohorts. In some embodiments, the present invention provides relationships between measured organ age and organ health and dysfunction relevant to multiple diseases of aging including heart disease, kidney disease, metabolic disease (e.g., metabolic syndrome, insulin resistance, type 2 diabetes, obesity), autoimmune disease, immune decline relevant to infectious disease, musculoskeletal disease (e.g., sarcopenia, osteopenia, osteoporosis), and neurodegeneration. In some embodiments, the present invention provides a brain aging assay that predicts the top quartile of brain agers to be nearly 4.4 times more likely to experience cognitive decline or dementia progression over a 5-year follow-up than the bottom quartile of brain agers in an independent cohort. In some embodiments, the present invention provides a heart aging assay that predicts the top quartile of heart agers to be 15 times more likely to experience congestive heart failure over a 15-year follow-up than the bottom quartile of heart agers in an independent cohort. In some embodiments, the present invention provides a minimally invasive framework to measure organ aging with proteomic aging signatures for 15 organs with biological and clinical significance to age-related morbidity and mortality.

In some embodiments, the present invention provides a method of identifying accelerated or slowed aging of an organ in a subject, comprising; obtaining a plasma sample from the subject; measuring the concentrations of two or more proteins from the organ in the plasma sample from the subject wherein the concentrations of the two or more proteins provides a biological age of the organ in health and/or disease; comparing the biological age of the organ to a chronological age of the subject, wherein a gap between the biological age of the organ and the chronological age of the subject identifies accelerated or slowed aging of the organ. In some embodiments, the accelerated or slowed aging provides a biomarker of dysfunction or disease in the organ. In some embodiments, the organ is a heart organ, a kidney organ, an immune system organ, a vascular system organ, a muscle organ, an intestine organ, adipose tissue, a liver organ, a lung organ, a pancreas organ, or a brain organ. In some embodiments, the sample is a bodily fluid sample, a whole blood sample, a buffy coat sample, a serum sample, a plasma sample, a urine sample, a saliva sample, a sweat sample, a sputum sample, a semen sample, a mucus sample, a lacrimal fluid sample, a lymph fluid sample, an amniotic fluid sample, an interstitial fluid sample, a cerebrospinal fluid sample, a feces sample, a tissue sample, an organ sample, a dried blood spot sample or a biopsy sample.

In some embodiments of the present invention, the measuring comprises use of a modified oligonucleotide aptamer-based assay. In some embodiments, the measuring comprises use of a multiplex immune-based assay. In some embodiments, the comparing comprises use of a machine learning model. In some embodiments, the machine learning model comprises Feature Importance for Biological Aging (FIBA). In some embodiments, the comparing comprises predicting LASSO regression-based chronological age and/or estimating LOWESS regression.

In some embodiments, the biological age is age in decades, years, months, weeks, days and/or hours. In some embodiments, the chronological age is age in decades, years, months, weeks, days and/or hours since birth. In some embodiments, a chronological age greater than a biological age identifies slowed aging of the organ. In some embodiments, a biological age greater than a chronological age identifies accelerated aging of the organ. In some embodiments, a biological age and a chronological age are the same age. In some embodiments, the accelerated aging comprises at least one pathophysiology in the organ. In some embodiments, the accelerated aging comprises two or more pathophysiologies in an organ.

In some embodiments the present invention comprises measuring the concentrations of two or more proteins from two or more organs in the sample from the subject wherein the concentrations of two or more proteins from two or more organs provides the biological ages of two or more organs in health and/or disease. In some embodiments, the two or more organs comprises five or more organs. In some embodiments, the two or more organs comprises ten or more organs. In some embodiments, the two or more of biological ages of the two or more organs is greater than the chronological age. In some embodiments, the accelerated aging comprises two or more pathophysiologies in two or more organs.

In some embodiments, the present invention comprises measuring the concentrations of two or more proteins from one or more cell types in an organ in a sample from a subject wherein the concentrations of the two or more proteins provides the biological age of the one or more cell types in the organ in health and/or disease.

In some embodiments of the present invention, the accelerated aging of an organ identified by the comparing directs one or more interventions to prevent and/or to reverse the accelerated aging of the organ in the subject. In some embodiments, the one or more interventions is one or more of a drug intervention, a drug prophylactic intervention, a drug curative intervention, a drug palliative intervention, a vaccine, a nutritional intervention, an educational intervention, a behavioral intervention, an environmental intervention, a surgical intervention, a radiologic-guided intervention, an applied energy intervention, an applied radiation intervention, a health systems intervention and/or a combination thereof.

In some embodiments the present invention comprises obtaining a sample from a subject after an intervention, measuring the concentrations of two or more proteins from an organ in a sample from a subject wherein the concentrations of two or more proteins provides a biological age of an organ in health and/or disease, and comparing the biological age of an organ to a chronological age of a subject, wherein a gap between the biological age of an organ and the chronological age of a subject identifies accelerated and slowed aging of an organ after an intervention. In some embodiments, the accelerated aging of an organ identified by the comparing directs one or more further interventions to prevent and/or reverse accelerated aging of an organ in a subject. In some embodiments, the intervention is drug. In some embodiments, the drug is a novel drug or a repurposed drug.

In some embodiments, the present invention provides a method of subject health maintenance, comprising: obtaining two or more interval samples from the subject, measuring the concentrations of two or more proteins from one or more organs in the two or more samples from the subject wherein the concentrations of the two or more proteins provides two or more biological ages of one or more organs in health and/or disease, comparing the biological ages of the one or more organs to a chronological age of the subject, wherein one or more gaps between the biological age of the one or more organs and the chronological age of the subject identifies accelerated and slowed aging of the one or more organs wherein the accelerated aging of the organ identified by the comparing directs one or more interventions to prevent and/or to reverse the accelerated aging of the one or more organs in the subject. In some embodiments, the comparing further comprises assessment of sex, albumin level, creatinine level, serum glucose level, C-reactive protein level, lymphocyte percent, mean red blood cell volume, red cell distribution width, alkaline phosphatase level, white blood cell count and DNA methylation of the subject. In some embodiments, the measuring comprises use of one or more organ-specific panels comprising two or more proteins. In some embodiments, the present invention provides the subject with information comprising the one or more gaps and/or the one or more interventions. In some embodiments, the information predicts future organ pathophysiology.

In some embodiments, the present invention provides a method to test an intervention to prevent and/or to reverse accelerated aging of an organ in a subject, comprising: obtaining a sample from the subject, measuring the concentrations of two or more proteins from the organ in the sample from the subject wherein the concentrations of the two or more proteins provides a biological age of the organ in health and/or disease, comparing the biological age of the organ to a chronological age of the subject, wherein a gap between the biological age of the organ and the chronological age of the subject identifies accelerated and slowed aging of organ, administering the intervention to the subject; obtaining one or more samples from the subject after the intervention, measuring the concentrations of two or more proteins from the organ in the two or more samples from the subject after the intervention wherein the concentrations of the two or more proteins provides a biological age of the organ in health and/or disease, and comparing the biological age of the organ to a chronological age of the subject before and after the intervention wherein a smaller gap between the biological age of the organ and the chronological age of the subject after the intervention identifies the intervention to prevent and/or reverse the accelerated aging of the organ.

### DESCRIPTION OF THE FIGURES

FIG. 1 shows identification of organ-enriched plasma proteins that model organ aging. a) Schematic illustrating measuring and validating organ-specific biological age. Organ-specific plasma proteins were identified by analyzing the RNA expression differences of plasma protein encoding genes among human organs in the Genotype-Tissue Expression (GTEx) protocol. A plasma protein was defined as organ-enriched if the gene encoding the protein was expressed at least 4-fold higher in one organ compared to any other organ. The mutually exclusive organ-specific protein sets were used to train bagged ensembles of least absolute shrinkage and selection operator (LASSO) chronological age predictors, or aging clocks, from 1,398 healthy individuals in the Knight-ADRC cohort. An “organismal” model, that used the non-organ-enriched proteins, and a “conventional” model, that used all panel proteins, were also trained. Models were tested in 4 independent cohorts: Covance (n=1,029), LonGenity (n=962), Stanford Aging and Memory Study (SAMS; n=192), Stanford ADRC (n=420). Models were also tested in Alzheimer's disease patients in the Knight-ADRC cohort (n=1,677). To assess whether plasma proteomic organ aging models captured physiologically relevant information beyond chronological age, measured organ ages were associated with age-related phenotypes after correcting for chronological age. To correct for chronological age, organ age gaps were calculated per cohort by subtracting predicted organ age from the LOWESS regression between predicted and chronological age. Organ age gaps were z-scored separately per organ model for downstream analyses to normalize for differences in model error. Organ age gaps were associated with diverse age-related phenotypes across the cohorts to assess their physiological relevance. b) Comparison of control age versus organ aging profile. Examples shows that individuals with the same conventional age gap have widely different distributions of organ age gaps. c) Pairwise correlation of organ age gaps in the cohorts showing that organ aging models capture distinct information. Distribution of the pairwise correlations is shown in inset histogram. The control age gap was highly correlated with the organismal age gap (r=0.98). d) Identification of extreme agers defined by a 2-standard deviation increase or decrease in at least one age gap. A kidney ager, heart ager, and multi-organ ager are provided as examples. e) Extreme agers were identified (23% of individuals) and clustered after setting age gaps below an absolute z-score of 2 to zero. The mean age gaps of kidney agers, heart agers, and multi-organ agers are shown.

FIG. 2 shows identification of organ-enriched plasma proteins. a) Proteins in plasma were mapped to specific organs of origin. Plasma proteins for which the gene encoding the protein was expressed at least 4-fold higher in one organ compared to any other organ were labelled as “organ-enriched” in keeping with the definition proposed by the Human Protein Atlas. To calculate organ-level gene expression, the maximum expression of sub-tissues in the Gene Tissue Expression Atlas (GTEx) bulk RNA-seq database was used further in keeping with the Human Protein Atlas. Expression aggregation into organ expression for gene CPLX1 is provided. b) Organ-wide expression for CPLX1 is shown. CPLX1 is expressed over 4-fold higher in the brain compared to any other organ and is therefore defined as organ-enriched. c) The organ-level fold-change distribution of SomaScan plasma protein encoding genes is shown. d) Organ-level expression of 843 organ-enriched plasma protein encoding genes is shown. The 843 genes correspond to 893 plasma protein epitopes measured on the SomaScan assay. Specific plasma proteins on the assay are quantified multiple times by different aptamers that target different epitopes of the same protein thereby accounting for the difference in the number of organ-specific plasma proteins and organ-enriched plasma protein encoding genes. 893/4,979 (17.9%) plasma proteins on the SomaScan are organ-enriched. e) Top significantly enrichment biological pathways of brain-enriched plasma proteins based on gProfiler are shown.

FIG. 3 shows protein quality control. a) Replicate measurement reproducibility assessed using: 1) Lin's concordance correlation coefficient (CCC) between replicate samples across SomaScan v4 and v4.1 assay versions (Somalogic); and 2) estimated coefficient of variation (CV) based on replicate samples in Candia, et. al. (Candia, J., Daya, G. N., Tanaka, T. et al. Assessment of variability in the plasma 7k SomaScan proteomics assay. Sci Rep 12, 17147 (2022). Proteins with high outlier values based on 3× the interquartile range for these metrics were removed.

FIG. 4 shows aging model training and testing. a) A bagged ensemble of least absolute shrinkage and selection operator (LASSO) aging model was trained for each of 11 major organs using the mutually exclusive organ-enriched proteins identified as inputs. An “organismal” aging model using the 3907 organ-nonspecific proteins and a “conventional” aging model using 4778 QC'ed proteins on the SomaScan assay were also trained. Models were trained from the 1,398 healthy individuals in the Knight-ADRC cohort. To reduce overfitting, the LASSO regularization parameter a was determined per bootstrap resampling by selecting the a that provided 95% performance. An individual's predicted age was defined as the average predicted age across the bootstrapped models. The entire model training scheme for a single example aging model is provided. b) Models were tested in 4 independent cohorts (Covance, LonGenity, Stanford-ADRC, SAMS). Age predictions from a single example aging model across test cohorts are shown.

FIG. 5 shows model age prediction and coefficients. a)-m) Aging model age prediction (i), average coefficients across bootstraps (ii), and top 15 coefficients (iii) are shown for the aging models in alphabetical order.

FIG. 6 shows a study design flow chart detailing embodiments of statistical tests performed in the course of development of the present invention.

FIG. 7 shows aging model characteristics and age gap calculation. a) Correlations between predicted vs. chronological age in healthy individuals in the training (Knight-ADRC) cohorts for aging models trained separately per sex, or trained with sex as a covariate. Models with sex as a covariate were used for the downstream analyses due to their performance to extend the generality of the findings and to reduce analytic complexity. b) Correlations between predicted vs chronological age in healthy individuals in the training (Knight-ADRC) and test (Covance, LonGenity, Stanford-ADRC, SAMS) cohorts for the aging models. The aging models significantly measured age across 5 independent cohorts. c) Display of the relationship between the number of proteins available for model training and the average number of proteins selected by the bootstrapped models. d) Display of the relationship between the average number of proteins selected by the bootstrapped models and the model accuracy in the train and test cohorts. e) Calculation of organ age gaps per cohort. An individual's age gap is defined as the difference between the individual's predicted age and the LOWESS regression curve between predicted and chronological age. f) Standard deviations of organ age gaps per cohort. Age gaps were z-score normalized separately per aging model for the downstream analyses to account for differences in model error and cohort effects.

FIG. 8 shows extreme organ agers in the population. a) Extreme agers were defined as individuals with a 2-standard deviation increase or decrease in at least one age gap. 23% of the population were identified as extreme agers. To visualize extreme agers age gaps were de-noised by setting values below absolute z-score of 2 to zero. De-noised age gaps are shown in the heatmap. b) Extreme ageotypes were defined based on kmeans clustering of individuals based on their de-noised age gaps. The mean z-scored age gap per ageotype is shown. c) The percentage of extreme agers is shown across the cohorts. d) A cross-cohort meta-analysis of associations between extreme ageotypes versus diagnosis of 9 major age-related diseases annotated in at least 2 independent cohorts, controlling for age and sex (logistic regression model: AgeGap˜Disease+Age+Sex). Log odds ratios and significance are shown. P-values were Benjamini Hochberg corrected. Asterisks represent q-value thresholds: * q<0.05; ** q<0.01; *** q<0.001. The strongest associations per disease are highlighted with black borders. e) A cross-cohort meta-analysis of associations between organ age gaps versus diagnosis of 9 major age-related diseases annotated in at least 2 independent cohorts, controlling for age and sex (linear model: AgeGap˜Disease+Age+Sex). Disease covariate effects and significance are shown. P-values were Benjamini Hochberg corrected. Asterisks represent q-value thresholds: * q<0.05; ** q<0.01; *** q<0.001. The strongest associations per disease are highlighted with black borders.

FIG. 9 shows measured organ age associated with multiple measures of health and disease. a) Forest plot displaying results from a cross-cohort meta-analysis of the association between the kidney age gap and hypertension history controlling for age and sex (linear model: AgeGap˜Disease+Age+Sex). Per cohort and total effect sizes, 95% confidence intervals, and Benjamini Hochberg total p-value (q-values) are shown. P-values were corrected based on 117 tests of organ age gap associations with major diseases (FIG. 8e). b) As in a), but for kidney age gap versus diabetes history. c) As in a), but for heart age gap versus atrial fibrillation or pacemaker history. d) As in a), but for heart age gap versus heart attack history. e) The bootstrap kidney aging model coefficients. Size of bubbles are scaled by the absolute value of the average model weight (absolute value of y-axis). f) Single-cell RNA expression of kidney aging model protein encoding genes in human kidney. Mean normalized expression values shown. g) Bootstrap heart aging model coefficients. Size of bubbles are scaled by the absolute value of the average model weight (absolute value of y-axis). h) Single-cell RNA expression of heart aging model protein encoding genes in human heart. Mean normalized expression values shown. i) Cox proportional hazard regression analysis in congestive heart failure risk controlling for age and sex (HeartFailureRisk˜AgeGap+Age+Sex), within 15 years in the LonGenity cohort showing 27 events out of 812 individuals. Hazard ratios, 95% confidence intervals, and Benjamini Hochberg corrected p-values (q-values) for z-scored organ age gaps are shown. j) Cox proportional hazard regression analysis in mortality risk controlling for age and sex (MortalityRisk˜AgeGap+Age+Sex), within 15 years in the LonGenity cohort showing 190 events out of 903 individuals. Hazard ratios, 95% confidence intervals, and Benjamini Hochberg corrected p-values (q-values) for z-scored organ age gaps are shown.

FIG. 10 shows plasma proteomic organ aging models versus established clinical markers of aging, health, and disease. a) Phenotypic Age (PhenoAge), a conventional clinical marker-based aging model, was calculated based on the 10 requisite clinical markers in the Covance cohort (n=1,026). PhenoAge-based age prediction is shown. b) The PhenoAge age gap was calculated and correlated with plasma proteomic organ aging model age gaps. Pairwise correlations are shown. c) Organ age gaps and the PhenoAge age gap were associated with 43 individual clinical markers of health and disease, controlling for age and sex (AgeGap˜Phenotype+Age+Sex). Phenotype covariate effect sizes and significance based on Benjamini Hochberg correction for the associations are shown. Asterisks represent q-value thresholds: *q<0.05; ** q<0.01; *** q<0.001.

FIG. 11 shows age gaps versus established clinical markers of aging, health, and disease. a) Organ age gaps and the PhenoAge age gap were associated with 43 individual clinical markers of health and disease controlling for age and sex (AgeGap˜Phenotype+Age+Sex) in the Covance cohort. Phenotype covariate effect sizes and significance based on Benjamini Hochberg correction for the associations are shown. Asterisks represent q-value thresholds: * q<0.05; ** q<0.01; *** q<0.001. b) U-shaped relationship between age and certain traits, including diastolic blood pressure, BMI, and alanine transaminase are shown.

FIG. 12 shows estimated glomerular filtration rate (EGFR) adjusted associations with disease. a) Kidney age gap associations with hypertension adjusted for EGFR in the LonGenity cohort. b) Kidney age gap associations with diabetes adjusted for EGFR in the LonGenity cohort.

FIG. 13 shows Feature Importance for Biological Aging (FIBA) to derive a cognition-associated brain aging model. a) Schematic of the application of permutation-based feature importance for biological aging (FIBA) to assess brain aging model protein contributions to the brain age gap association with cognition and chronological age prediction accuracy: 1) the brain aging model was trained from heathy individuals in the Knight-ADRC cohort; 2) the brain aging model was evaluated by determining its association with chronological age and cognition (CDR-Gobal); 3) expression values for one protein were permuted; 4) the model was re-evaluated to determine the importance of that protein in the model's association with chronological age and cognition. FIBA scores are calculated as the difference in association effect sizes before and after permutation; and 5) steps 1˜4 were repeated for the bootstrapped models, for the model proteins, and 3 times per protein. FIBA results across replicates and bootstraps are averaged and plotted. Significance is determined based on the proportion of positive FIBA scores among bootstraps. A protein is defined as significant (FIBA+) if <5% of its FIBA scores across bootstraps is negative. Only proteins with nonzero coefficients in at least 100/500 bootstraps were considered. FIBA+ brain aging model proteins were used to train a new cognition-optimized brain aging model (CognitionBrain) from healthy individuals in the Knight-ADRC cohort. b) Forest plots displaying results from cross-cohort meta-analyses of associations between the CognitionBrain, Brain, and Control age gaps versus Alzheimer's disease diagnosis controlling for age and sex (linear model: AgeGap˜Alzheimer's+Age+Sex). Per cohort and total effect sizes, 95% confidence intervals, and total p-values are shown. Feature optimization with FIBA improves model age gap associations with Alzheimer's disease. c) CognitionBrain age gaps were associated with brain MRI volume in the Stanford-ADRC and SAMS cohorts (n=469). CognitionBrain associations with individual brain region volumes are shown controlling for estimated total intracranial volume, age, and sex (CognitionBrainAgeGap˜eTIV-corrected-RegionVolume+Age+Sex). Bubbles are sized by the Benjamini Hochberg corrected p-values (q-value) of the brain region volume covariate. d) Pairwise-correlations between the CognitionBrain age gap and conventional biomarkers of AD i.e., plasma pTau-181 and AD polygenic risk score are shown.

FIG. 14 shows the role of brain aging in cognitive decline and Alzheimer's disease. a) Results from permutation-based feature importance for biological aging (FIG. 13a). Brain aging model proteins were assessed for their contribution to the brain age gap association with dementia (y-axis) and chronological age prediction accuracy (x-axis). Permutation of proteins such as CNDP1, NPTXR, CPLX1 reduced the brain age gap association with dementia (FIBA+), while premutation of proteins such as PIANP, PP3R1 strengthened the association with dementia (FIBA−). FIBA+ brain aging model proteins were used to train a de novo cognition-optimized brain aging model (CognitionBrain) from cognitively unimpaired individuals in the Knight-ADRC cohort. b) CognitionBrain aging model: i) Age measurement in the cohorts; and ii) The bootstrap aging model coefficients. Size of bubbles are scaled by the absolute value of the average model weight (absolute value of y-axis). c) Forest plot displaying results from a cross-cohort meta-analysis of the association between the CognitionBrain age gap versus Alzheimer's disease diagnosis controlling for age and sex (linear model: AgeGap˜Disease+Age+Sex). Per cohort and total effect sizes, 95% confidence intervals and total p-value are shown. d) Cox proportional hazard model of dementia progression risk including biomarkers of AD and predictors of cognitive decline as covariates (2 pt increase in CDR-Sum of Boxes˜CognitionBrainAgeGap+CDR-Global+PlasmaPTau181+ADPolygenicRiskScoreAD+Age) within 5 years in the Stanford-ADRC cohort showing 48 events out of 325 individuals. Hazard ratios, 95% confidence intervals, and p-values for the covariates are shown. e) Cumulative incidence plot derived from hazard model in f). Shown are predicted trajectories based on changing levels of the 2 significant biomarker covariates, CognitionBrainAgeGap and PlasmaPTau181, while other covariates remain constant. Individuals with biomarker levels 2 standard deviations above average had over a 75% probability of dementia progression, while individuals with levels 2 standard deviations below average had a 10% probability of dementia progression within 5 years in the Stanford-ADRC cohort. f) Single-cell RNA expression of CognitionBrain aging model protein encoding genes in human brain. 47/49 model proteins were expressed and mean normalized expression values are shown. Nine of the top 10 model proteins by absolute weight were expressed. Top model proteins and proteins in the GO: CC synapse pathway are highlighted. g) Changes with age and AD of top CognitionBrain proteins across tissues (plasma, brain) and molecular layers (protein, bulk RNA, single-cell RNA). Proteins with significant changes in both fluid and solid tissues are shown. Plasma and CSF changes with age and AD were assessed using the linear model: Protein˜Age+AD+Sex. Brain protein and bulk RNA changes with AD were derived from Johnson et. al. 202255 supplementary tables. Brain single-cell RNA changes with AD were provided by Michael Haney, Stanford University.

FIG. 15 shows CognitionBrain age gaps associated with brain MRI volume in the Stanford-ADRC and SAMS cohorts (n=469). b) The publicly available brain MRI based aging model, BARACUS Brain-Age, was tested in the Stanford-ADRC and SAMS cohorts. The age prediction image from the original publication Liem et. al. 201721 is shown. An age prediction ceiling is observed. c) Age prediction using BARACUS in the Stanford-ADRC and SAMS cohorts is shown. An age prediction ceiling is observed in both the original publication and when tested in Stanford cohorts. d) The publicly available brain MRI based aging model, brainageR, was tested in the Stanford-ADRC and SAMS cohorts. The age prediction image from the github is shown. No age prediction ceiling is observed. e) The age prediction using brainageR in the Stanford-ADRC and SAMS cohorts is shown. No age prediction ceiling is observed. f) The correlation between the CognitionBrain age gap and brainageR age gap is shown.

FIG. 16 shows changes in the aging vasculature and extracellular matrix that precede dementia incidence in cognitively healthy individuals. a) The FIBA optimization framework was applied to other organ aging models to measure how aging of other organs contributes to brain aging phenotypes. As with the brain aging model, CDRGLOB FIBA was applied to the aging models using the Knight-ADRC. b) The CognitionArtery, CognitionBrain, CognitionOrganismal, and CognitionPancreas age gap associations with AD replicated in both ADRCs, and the 4 aging models were used to test peripheral versus central contributions to cognitive decline. c) Associations between z-scored organ age gaps and a composite score of overall cognition, controlling for age and sex (linear model: AgeGap˜Cognition+Age+sex) in the LonGenity cohort (n=888). Effect sizes, 95% confidence intervals, and p-values for z-scored organ age gaps are shown. d) Cox proportional hazard regression analysis in risk of conversion from healthy to mild cognitive impairment (CDR-Global 0->>=0.5), controlling for age and sex (MCIRisk˜AgeGap+Age+Sex), within 15 years in the Knight-ADRC cohort showing 226 events out of 940 individuals. Hazard ratios, 95% confidence intervals, and p-values for z-scored organ age gaps are shown. e) Aging trajectories of top 10 weighted model proteins as locally weighted scatterplot smoothing (LOWESS) curves. Proteins were z-scored, then shifted so that the proteins start at zero. Top CognitionOrganismal proteins change with age earliest and at the highest rate. f) Changes with age of cognition-optimized aging model proteins (linear model: Protein˜Age+Sex). Age effect and negative log₁₀ Benjamini Hochberg corrected p-values (q-values) are shown. Size of bubbles are scaled by the absolute value of the average model weight. g) Single-cell RNA expression of top 5 CognitionOrganismal protein encoding genes in human brain vasculature. Mean normalized expression values and fraction of cells expressing the genes are shown. Also shown are cell composition differences in the human brain vasculature between Alzheimer's and control participants based on the same single-cell RNA-sequencing dataset. h) Model of age-related cellular degradation of the human brain vasculature reflected in the plasma proteome. i) StringDB protein-protein-interaction network of CognitionArtery and interacting proteins, as well as pathway enrichment of these proteins. j) Model of age-related vascular calcification and extracellular matrix alterations reflected in the plasma proteome.

FIG. 17 shows Feature Importance for Biological Aging (FIBA) plots for aging models in relation to cognition. a) Permutation-based feature importance for biological aging (FIG. 13a) was applied to aging models in relation to cognition to assess peripheral versus central contributions to brain aging and cognitive decline. For each aging model proteins were assessed for their contributions to the age gap association with cognition (CDR-Global) (y-axis) and chronological age prediction accuracy (x-axis). Proteins for which permutation reduces the age gap association with cognition were termed FIBA+, while proteins for which permutation strengthens the age gap association with dementia were termed FIBA−. FIBA+ proteins were used to train new cognition-optimized aging models from healthy individuals in the Knight-ADRC cohort. FIBA results for the aging models are shown in alphabetical order.

FIG. 18 shows cognition-optimized aging model associations with age and Alzheimer's disease across cohorts. a) FIBA+ proteins from each aging model were used to train cognition-optimized aging models from healthy individuals in the Knight-ADRC cohort. Correlations between predicted vs. chronological age in healthy individuals in the training (Knight-ADRC) and test (Covance, LonGenity, Stanford-ADRC, SAMS) cohorts for the aging models are shown. Aging models significantly measured age across 5 independent cohorts. Cognition-optimized aging models predicted chronological age slightly worse than their non-optimized counterparts as expected given subsetting of proteins. b) Pairwise correlation of the model age gaps in the cohorts. Cognition-optimized aging models predicted similar age gap measured with their non-optimized models. c) Model age gap associations with Alzheimer's disease diagnosis controlling for age and sex (AgeGap˜Alzheimer's+Age+Sex) in the Knight-ADRC cohort are shown. Effect sizes, 95% confidence intervals, and p-values for the Alzheimer's covariate are provided. Despite decreased associations with chronological age, cognition-optimized models show substantially stronger associations with Alzheimer's disease. d) As in c), but in the Stanford-ADRC cohort.

FIG. 19 shows dementia-optimized aging model associations with cognitive function in non-cognitively impaired individuals across cohorts. a) Associations between organ age gaps and cognitive function, controlling for age, sex, and education (AgeGap˜OverallCognitionComposite+Age+Sex+Education) in the healthy LonGenity cohort (n=888). b) Associations between organ age gaps and memory, controlling for age, sex, and education (AgeGap˜AssociativeD′+Age+Sex+Education) in the healthy SAMS cohort (n=160).

FIG. 20 shows Mapping CognitionOrganismal and CognitionArtery proteins to human organs and cell types. a) The organ sources of highly weighted CognitionOrganismal proteins were investigated by analyzing their expression levels in the Gene Tissue Expression Atlas (GTEx) bulk RNA-seq database. Organ-level expression of pleiotrophin (PTN), transgelin (TAGLN), WNT1 inducible signaling pathway protein 2 (WISP2), and chordin like 1 (CHRDL1) are shown. These genes were highly expressed in the arteries and brain. b) Single-cell RNA expression of highly weighted CognitionOrganismal proteins in human vasculature. Mean normalized expression values and fraction of cells expressing the genes are shown. c) Single-cell RNA expression of highly weighted CognitionArtery and StringDB-based “interacting” proteins in human vasculature. Mean normalized expression values and fraction of cells expressing the genes are shown.

FIG. 21 shows an embodiment of a machine learning custom protein quality control pipeline. a) Principal component analysis (PCA) of the healthy human plasma proteome. The first 2 PCs are shown. Healthy individuals across cohorts differ in PC space, which suggests technical cohort effects. b) First QC step: reduce protein measurement noise. Replicate measurement reproducibility assessed using: 1) Lin's concordance correlation coefficient (CCC) between replicate samples across SomaScan v4 and v4.1 assay versions (Somalogic) and 2) estimated coefficient of variation (CV) based on replicate samples in Candia, et. al. (Candia, J., Daya, G. N., Tanaka, T. et al. Assessment of variability in the plasma 7k SomaScan proteomics assay. Sci Rep 12, 17147 (2022). Proteins with high outlier values based on 3 times the interquartile range for these metrics were removed. c) Second QC step: reduce cohort effects. Proteins that differ between healthy individuals across cohorts with an effect size larger than that from biological sex are presumed to be driven by sample handling/technical factors (i.e., blood processing, freezing, thawing, etc.). For each protein, a linear model Protein level˜Sex+Age+Cohort was tested. A cohort effect size that is 5 standard deviations above or below the mean sex effect is filtered out. d) An example protein with a large cohort effect. Protein levels per cohort are shown. e) An example protein that is stable across cohorts. Protein levels per cohort are shown. f) After both QC steps, ˜1,000/5,000 proteins are filtered out.

FIG. 22 shows Feature Importance for Biological Aging (FIBA) to derive a cognition-associated brain aging model. a) Schematic of the application of permutation-based feature importance for biological aging (FIBA) to assess brain aging model protein contributions to the brain age gap association with cognition and chronological age prediction accuracy: 1) the brain aging model was trained from heathy individuals in the Knight-ADRC cohort; 2) the brain aging model was evaluated by determining its association with chronological age and cognition (CDR-Gobal); 3) expression values for one protein were permuted; 4) the model was re-evaluated to measure the importance of that protein in the model's association with chronological age and cognition. FIBA scores are calculated as the difference in association effect sizes before and after permutation; and 5) steps 1˜4 were repeated for the bootstrapped models, for the model proteins, and 3 times per protein. FIBA results across replicates and bootstraps are averaged and plotted. Significance is determined based on the proportion of positive FIBA scores among bootstraps. A protein is defined as significant (FIBA+) if <5% of its FIBA scores across bootstraps are negative. Only proteins with nonzero coefficients in at least 100/500 bootstraps were considered. FIBA+brain aging model proteins were used to train a new cognition-optimized brain aging model (CognitionBrain) from healthy individuals in the Knight-ADRC cohort.

FIG. 23 shows Olink based aging clocks. a) Correlation between Olink protein levels and SomaScan protein levels for NPPB and REN. b) Heart aging model and Kidney aging model based on Olink heart and kidney proteins. Age prediction is shown. c) Association between Olink heart age gap and atrial fibrillation, and association between Olink kidney age gap and diabetes

FIG. 24 shows linear and non-linear changes with age in the plasma proteome. a) Age distributions per cohort. b) Cohort normalization to assess plasma proteome changes with age, independent of cohort age distribution. Cohort normalization was not applied for training and assessing aging models across cohorts. c) Change with age in the plasma proteome (Protein˜Age+Sex). Age effects and transformed Benjamini Hochberg adjusted p-values shown. d) Locally weighted scatterplot smoothing (LOWESS) curves for aging plasma proteome shown. e) LOWESS curves plotted as heatmap. f) Example proteins that change non-linearly with age.

### DEFINITIONS

Throughout the specification and claims, the following terms take the meanings explicitly associated herein, unless the context clearly dictates otherwise. The phrase “in some embodiments” as used herein does not necessarily refer to the same embodiment, though it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described unless the context clearly dictates otherwise. In addition, throughout the specification, the meaning of “a,” “an,” and “the” include plural references. The meaning of “in” includes “in” and “on.”

The term “one or more,” as used herein, refers to a number higher than one. For example, the term “one or more” encompasses any of the following: two or more, three or more, four or more, five or more, six or more, seven or more, eight or more, nine or more, ten or more, eleven or more, twelve or more, thirteen or more, fourteen or more, fifteen or more, twenty or more, fifty or more, 100 or more, or an even greater number.

The term “one or more but less than a higher number,” “two or more but less than a higher number,” “three or more but less than a higher number,” “four or more but less than a higher number,” “five or more but less than a higher number,” “six or more but less than a higher number,” “seven or more but less than a higher number,” “eight or more but less than a higher number,” “nine or more but less than a higher number,” “ten or more but less than a higher number,” “eleven or more but less than a higher number,” “twelve or more but less than a higher number,” “thirteen or more but less than a higher number,” “fourteen or more but less than a higher number,” or “fifteen or more but less than a higher number” is not limited to a higher number. For example, the higher number can be 10,000, 1,000, 100, 50, etc. For example, the higher number can be approximately 50 (e.g., 50, 49, 48, 47, 46, 45, 44, 43, 42, 41, 40, 39, 38, 37, 36, 35, 34, 33, 32, 31, 32, 30, 29, 28, 27, 26, 25, 24, 23, 22, 21, 20, 19, 18, 17, 16, 15, 14, 13, 12, 11, 10, 9, 8, 7, 6, 5, 4, 3 or 2).

As used herein, the term “circulating tumor DNA” (or “ctDNA”) is tumor-derived DNA that is circulating in the peripheral blood of a patient. ctDNA is of tumor origin and originates directly from the tumor or from circulating tumor cells (CTCs), which are viable, intact tumor cells that shed from primary tumors and enter the bloodstream or lymphatic system. The term “cf-tDNA” refers to cell free tumor DNA in a circulating or non-circulating body fluid.

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid.” The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, etc.

The term “aptamer” as used herein refers to a compound comprising an oligonucleotide molecule that can bind to a target, including small molecules, proteins, and peptides among others, with high affinity and specificity. Aptamers may assume a variety of shapes due to their propensity to form helices and single-stranded loops. An aptamer is a set of copies of one type or species of nucleic acid molecule that has a particular nucleotide sequence. An aptamer can include any suitable number of nucleotides, including any number of chemically modified nucleotides. Aptamers refers to more than one such set of molecules. Different aptamers can have either the same or different numbers of nucleotides. Aptamers may be DNA or RNA and may be single stranded, double stranded, or contain single, double, or triple stranded regions. Aptamers can comprise chemically modified nucleic acids and can include higher ordered structures.

The term “gene” refers to a nucleic acid (e.g., DNA or RNA) sequence that comprises coding sequences necessary for the production of an RNA, or of a polypeptide or its precursor. A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a “gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural, and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, a “diagnostic” test application includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and/or determining the effect of a treatment on a subject with a disease or condition. For example, a diagnostic test can be used for detecting the presence or likelihood of a subject contracting a neoplasm or the likelihood that such a subject will respond favorably to a compound (e.g., a pharmaceutical, e.g., a drug) or other treatment.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

As used herein, the terms “patient” or “subject” refer to organisms to be subject to various tests described herein. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided herein. As such, the present disclosure provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including racehorses), and the like.

As used herein, the term “kit” refers to any delivery system for delivering materials. In the context of reaction assays, such delivery systems include systems that allow for the storage, transport, or delivery of reaction reagents (e.g., aptamers, antibodies, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a sub-portion of the total kit components. The containers may be delivered to the intended recipient together or separately. For example, a first container may contain an enzyme for use in an assay, while a second container contains an aptamer and/or an antibody. The term “fragmented kit” is intended to encompass kits containing Analyte specific reagents (ASR's) regulated under the Federal Food, Drug, and Cosmetic Act, but are not limited thereto. Indeed, any delivery system comprising two or more separate containers that each contains a sub-portion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “proteomic information” refers to information pertaining to a proteome including, but not limited to, proteins, peptides, polypeptides, protein expression, phenotypes correlating to proteomic biomarkers, etc. Proteomic information may include use of the OLink platform. OLink proteomics, includes immunoassay, extension, preamplification, and detection by microfluidic qPCR, enables detection, visualization and quantification of individual proteins, protein modifications and protein interactions and has a high multiplex ability.

As used herein, the terms “sample,” “test sample,” and “biological sample” refer to a sample containing or suspected of containing a proteomic biomarker of the present disclosure. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis. In a particular example, the source is a mammalian (e.g., human) bodily substance (e.g., bodily fluid, blood such as whole blood, buffy coat, serum, plasma, urine, saliva, sweat, sputum, semen, mucus, lacrimal fluid, lymph fluid, amniotic fluid, interstitial fluid, cerebrospinal fluid, feces, tissue, organ, one or more dried blood spots, biopsy or the like). The sample may be a liquid sample or a liquid extract of a solid sample. In some embodiments, the source of the sample may be an organ or tissue, such as a biopsy sample and/or an endoscopic brushing sample (e.g., endoscopic esophageal brushing sample), which may be solubilized by tissue disintegration/cell lysis. Samples can be obtained by any number of methodologies. Cell free or substantially cell free samples can be obtained by subjecting the sample to various techniques including but are not limited to, centrifugation and filtration. In some embodiments, one or more proteins are isolated from a sample (e.g., a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample).

### DETAILED DESCRIPTION OF THE DISCLOSURE

Provided herein are minimally invasive compositions, methods, systems, kits and uses for biomarkers derived from the plasma proteome that identify, predict, and monitor organ health, aging, dysfunction and disease in humans.

Aging results in organism-wide deterioration of tissue structure and function that drastically increases risk of most chronic diseases¹. Studies of the molecular changes that occur with aging across multiple organs in mice have identified unique molecular aging trajectories and timings²⁻⁵, and susceptibility and resilience to diseases of aging in specific organs such as the brain, heart, and kidney varies substantially across the population⁶. How human organs change molecularly with age is poorly understood. The molecular characterization of human organ aging is of critical importance to address the massive global disease burden of aging. and will revolutionize patient care, preventative medicine, and drug development⁷. Preclinical studies demonstrate that rejuvenating interventions affect organs differently^{3, 8, 9}. To translate preclinical data into transformative medicines, it is critical to accurately measure aging across the body and to account for the diversity of human aging not only across but also within individuals.

While many methods to measure molecular aging in humans have been developed¹⁰⁻¹⁸, most provide just a single composite measure or value of aging for the whole body that is difficult to interpret given the complexity of human aging trajectories. No single method to date predicts aging outcomes in all organs. Several recent methods use clinical chemistry markers which include markers of organ function^{10, 16, 19}. However, these methods only generate a single composite score for the whole body and contain many markers with low organ specificity making them difficult to interpret for organ-specific aging. Methods to measure brain aging have used MRI-based brain volume and functional connectivity measurements but are costly, time-consuming, and do not provide molecular insights^{20, 21}. Certain organ-specific plasma proteins non-invasively assess aspects of organ health, such as troponin T for heart damage²² and alanine transaminase for liver damage²³. To improve on conventional biomarkers of organ specific aging, in some embodiments the present invention provides quantification of organ-specific proteins in plasma that are minimally invasive, and that track human aging for specific and diverse organs. In some embodiments, compositions, methods, systems, and kits of the present invention measure aging in 11 major organs across the human lifespan. Experiments conducted in the course of development of the present invention disclosed that nearly 20% of the population show accelerated age in at least one organ, and that 1.7% of the population are multi-organ agers. In turn, accelerated organ aging confers 20-50% higher mortality risk, and organ-specific diseases contribute to faster aging of those organs. In some embodiments, individuals with accelerated heart aging experience a 250% increased heart failure risk. In some embodiments, accelerated brain and vascular aging predict Alzheimer's disease progression independently from and as significantly as plasma pTau-181, a validated biomarker for Alzheimer's disease. In some embodiments, biomarker panels of the present invention identify vascular calcification, extracellular matrix alterations, and synaptic protein shedding that predict early cognitive decline. Accordingly, the present invention provides compositions, methods, systems, kits and uses to quantify organ-specific aging, and to predict organ-specific aging and multi-organ aging.

In some embodiments, companion compositions, methods, systems, kits, and uses of the present invention comprise organ-specific plasma protein age biomarkers to direct therapeutic interventions. In some embodiments, organ-age directed therapeutic interventions prevent accelerated organ age. In some embodiments, organ-age directed therapeutic interventions reverse accelerated organ age. In some embodiments, organ-age directed therapeutic interventions prevent one or more organ specific diseases and/or conditions. In some embodiments, organ-age directed therapeutic interventions treat and/or cure one or more organ specific disease and/or conditions. In some embodiments, an organ-age directed therapeutic intervention of the present invention is a drug prophylactic intervention, a drug curative intervention or a drug palliative intervention, a vaccine, a nutritional intervention, an educational intervention, a behavioral intervention, an environmental intervention, a surgical intervention, a radiologic-guided intervention, an applied energy or applied radiation intervention, a health systems intervention, or a combination of organ-directed therapeutic interventions. In some embodiments, an organ-age directed intervention of comprises a novel drug. In some embodiments, an organ-age directed intervention comprises a repurposed drug. In some embodiments, the repurposed drug is rapamycin to preserve intestinal function. (Juricic, P., Lu, YX., Leech, T. et al. Long-lasting geroprotection from brief rapamycin treatment in early adulthood by persistently increased intestinal autophagy. Nat Aging 2, 824-836 (2022). In some embodiments, the repurposed drug is metformin to alleviate age-induced neurocognitive deficit via amelioration of neuroinflammation, attenuation of oxidative stress, reduction of apoptosis and promotion of synaptic plasticity. (Ameen, O., Samaka, R. M. & Abo-Elsoud, R. A. A. Metformin alleviates neurocognitive impairment in aging via activation of AMPK/BDNF/PI3K pathway. Sci Rep 12, 17084 (2022). In some embodiments, the repurposed drug is acarbose to decrease lesions of the heart and kidney, and to suppress cardiac and renal pathology associated with increasing age. (Gupta S, Jiang Z, Ladiges W. The antidiabetic drug acarbose suppresses age-related lesions in C57BL/6 mice in an organ dependent manner. Aging Pathobiol Ther. 2021 Jun. 29; 3 (2):41-42, Jiang, Z., Wang, J., Imai, D. et al. Short term treatment with a cocktail of rapamycin, acarbose and phenylbutyrate delays aging phenotypes in mice. Sci Rep 12, 7300 (2022)). In some embodiments, the repurposed drug is dasatinib to attenuate adipose tissue inflammation, ameliorate metabolic function in old age, and to ameliorate age-dependent intervertebral disc degeneration. (Islam M T, Tuday E, Allen S, Kim J, Trott D W, Holland W L, Donato A J, Lesniewski L A. Senolytic drugs, dasatinib and quercetin, attenuate adipose tissue inflammation, and ameliorate metabolic function in old age. Aging Cell. 2023 February; 22 (2):e13767, Novais, E. J., Tran, V. A., Johnston, S. N. et al. Long-term treatment with senolytic drugs Dasatinib and Quercetin ameliorates age-dependent intervertebral disc degeneration in mice. Nat Commun 12, 5213 (2021).) In some embodiments, the nutritional intervention is taurine to improve diverse organ functions and increase health span. (Singh P et al. Taurine deficiency as a driver of aging. Science. 2023 Jun. 9; 380(6649):eabn9257.) In some embodiments, the nutritional intervention is urolithin A to induce mitophagy, prolongs lifespan and increase muscle function. (Ryu, D., Mouchiroud, L., Andreux, P. et al. Urolithin A induces mitophagy and prolongs lifespan in C. elegans and increases muscle function in rodents. Nat Med 22, 879-888 (2016).) In some embodiments, the nutritional intervention is glucosamine to rejuvenate epidermal and dermal markers associated with age. (Gueniche A, Castiel-Higounene I. Efficacy of Glucosamine Sulphate in Skin Ageing: Results from an ex vivo Anti-Ageing Model and a Clinical Trial. Skin Pharmacol Physiol. 2017; 30(1):36-41.)

In some embodiments, the organ-age directed interventions treat Alzheimer's disease and cognitive decline, or symptoms thereof. For example, Alzheimer's disease interventions include administration of therapeutic monoclonal antibodies against Aβ oligomers (e.g., aducanumab, lecanemab, BAN2401, solanezumab, see, also U.S. Pat. Nos. 7,811,563, 7,780,963, and 7,731,962); administration of medications (e.g., cholinesterase inhibitors (e.g., aricept (e.g., donepezil), exalon (e.g., rivastigmine), razadyne (e.g., galantamine)); NMDA antagonists (e.g., memantine (e.g., namenda) and memantine/donepezil combinations (e.g., namzaric); drugs targeting Aβ oligomers or oligomer formation (e.g., ALZ-801); metformin); behavior monitoring or modification; treatments for sleep changes; mindfulness and cognitive training, dietary changes; and the like. Furthermore, the intervention may be a customized treatment, for example, customized T cells that target Alzheimer's disease (see, e.g., U.S. Patent Publication 2022/0170908).

### EXPERIMENTAL EXAMPLES

### Example 1—Organ-Specific Plasma Proteins can Model Organ Aging

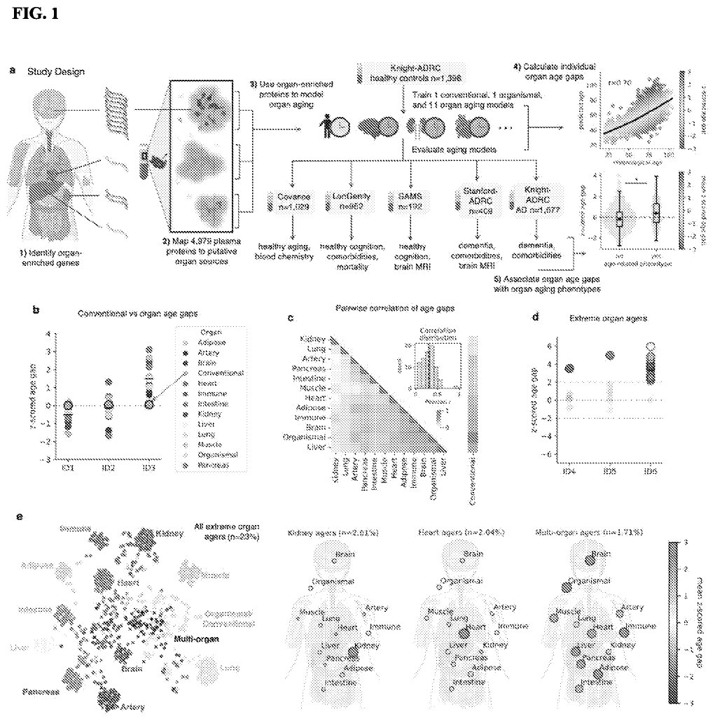

We measured 4,979 proteins in 5,678 participants across 5 independent cohorts (Table 1) and mapped the organ-specific plasma proteome used to train models of organ aging (FIG. 1a). We mapped the organ-specific plasma proteome using human organ bulk RNA-seq data from the Genotype-Tissue Expression (GTEx) project²⁴. We classified genes as “organ enriched” if they were expressed at least 4 times higher in one organ compared to any other organ according to the definition proposed in the Human Protein Atlas²⁵.

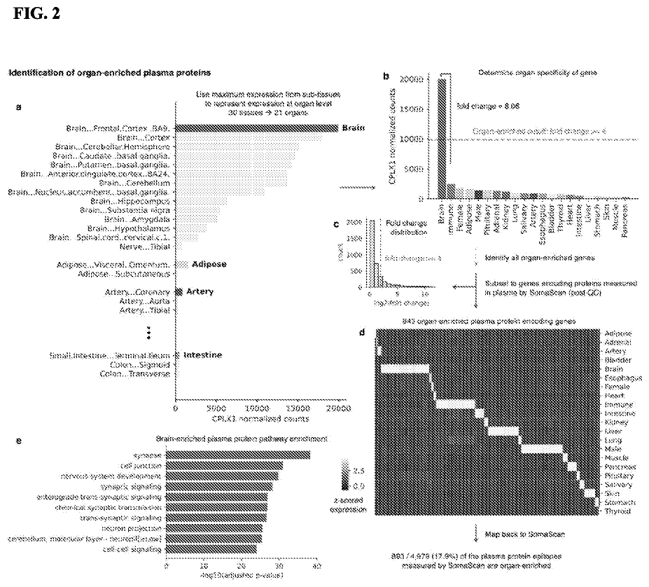

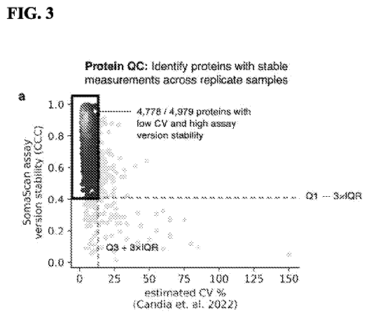

(FIG. 2, Tables 2-3). We annotated the 4,979 human proteins measured by the SomaScan assay and identified 893 (18%) proteins that met this definition, with the highest number from the brain consistent with the specialization and unique gene expression pattern of the central nervous system²⁵. We performed additional quality control to remove proteins with high coefficients of variation or low correlations between 2 different versions of the SomaScan assay present across our cohorts, with 4,778 proteins (856 organ enriched, 17.9%) remaining used for downstream analysis (FIG. 3, Tables 4-5).

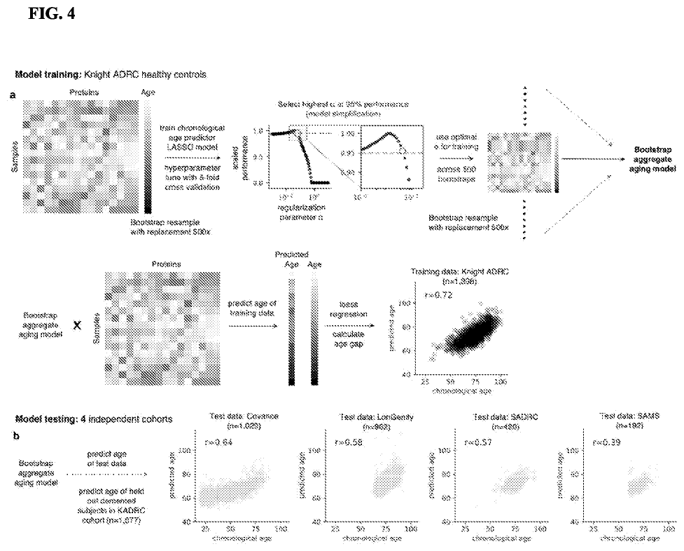

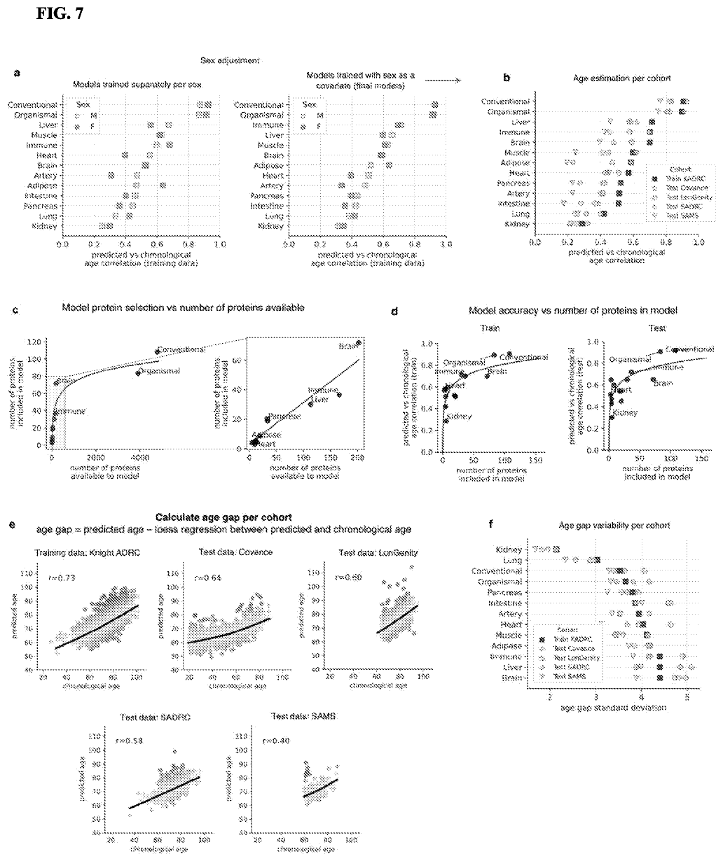

Plasma proteins may be used to train machine learning models to measure chronological age in independent cohorts^{13, 14}. For each individual, an aging model produces an “age gap”, a measure of that individual's biological age relative to other same aged peers based on their molecular profile^{11, 12, 19, 26-29} (FIG. 1a). Studies show associations between age gaps and mortality risk or other age-related phenotypes^{14, 18, 30, 31}, indicating that the age gap contains information relevant to biological aging. Accordingly, we trained a bagged ensemble of least absolute shrinkage and selection operator (LASSO) aging models for 11 major organs using the mutually exclusive organ-enriched proteins we identified as inputs (FIG. 1a, FIG. 4a-b, FIG. 7, Tables 6-7). We restricted analyses to adipose tissue, artery, brain, heart, immune tissue, intestine, kidney, liver, lung, muscle, and pancreas because of their well-understood contributions to diseases of aging and the availability of relevant age-related phenotype data in the tested cohorts. We also trained an “organismal” aging model using 3,907 organ-nonspecific plasma proteins as inputs to compare the contribution of specific organs to an organ-shared aging signature, and a “conventional” proteomic aging model using the 4,778 proteins to compare the organ aging models to a global plasma proteomic aging signature^{32, 33}. We trained models in 1,398 healthy participants from the Knight-ADRC cohort (mean age=75, age range=27-104) and then tested these models in 4 fully independent cohorts, and in held-out test participants with dementia in the Knight-ADRC. (FIG. 1a, Extended Data FIG. 4-6). The 11 organ aging models and the organismal model significantly measured organ age in the 5 cohorts after multiple test correction (FIG. 7b).

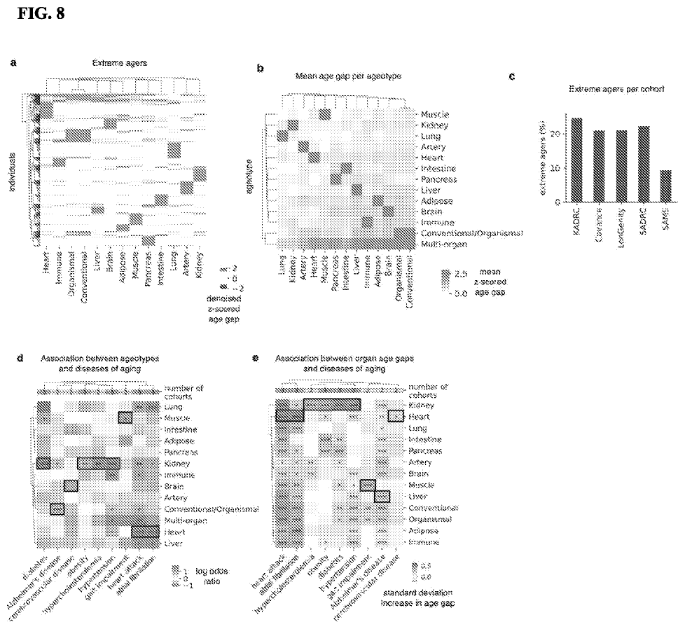

We observed across the cohorts that individuals with the same conventional age gap had diverse organ aging profiles (FIG. 1b). At the population level, this resulted in a low-to-moderate correlation between the age gaps of different organs (mean pairwise Pearson r=0.29, FIG. 1c). While organ aging is correlated, the majority of variance in one organ age gap is not explained by others, with the exception of the organismal and conventional age gaps which were highly correlated. Further, we observed that some individuals had extreme aging in one or more organs relative to the general population (FIG. 1d). We scored individuals across the cohorts as outliers for a given organ age gap using a 2-standard deviation cutoff and clustered individuals into extreme aging types (e-ageotypes) (FIG. 1e, FIG. 8a-c). Although extreme aging in one organ could co-occur with extreme aging in other organs, we observed segregation into distinct organ e-ageotypes. Approximately 18.4% of individuals had a highly organ-specific e-ageotype that was dominated by the aging of only 1 organ. Approximately 1.7% of individuals showed extreme aging in multiple organs. The only multi-organ e-ageotype identified through unbiased clustering was defined by extreme adipose, brain, conventional, heart, immune, liver, and organismal age gaps. These observations indicate that organ age gaps capture unique aging information that may have impacts for organ-specific biological aging and diseases of aging.

### Example 2—Proteomic Organ Age is Associated with Multiple Measures of Health and Disease

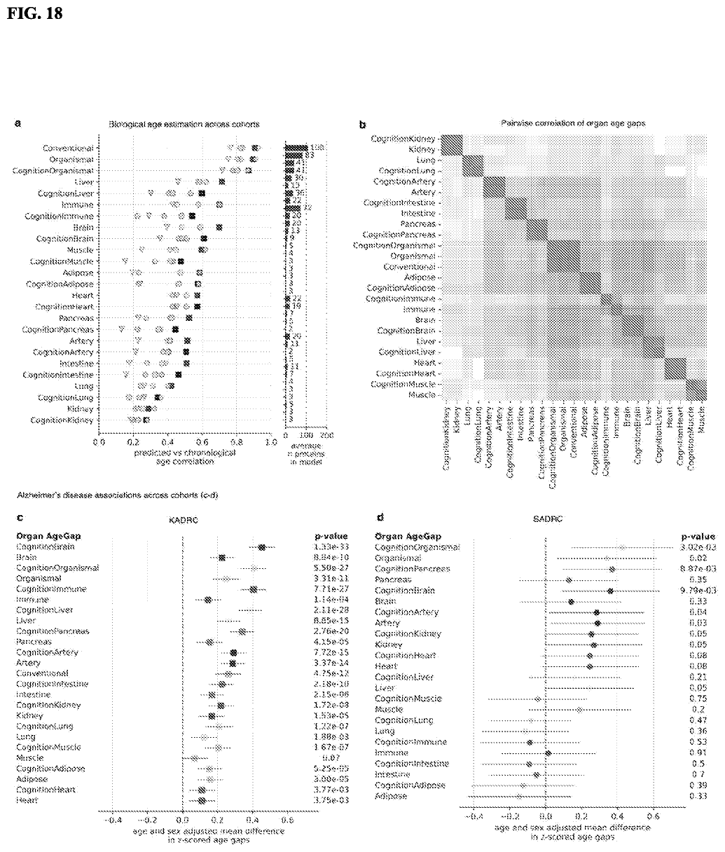

To assess the relationship between organ age and biological aging, we tested whether organ e-ageotypes were associated with 9 age-related disease states with sufficient data in at least 2 independent cohorts; Alzheimer's disease, atrial fibrillation, cerebrovascular disease, diabetes, heart attack, hypercholesterolemia, hypertension, obesity, and gait impairment. Organ e-ageotypes were associated with specific disease states with known high impact on their respective organs (23/117, 20%, associations significant in a meta-analysis after multiple testing correction, FIG. 8d, Table 8). The kidney ageotype was the most significantly associated with metabolic diseases (diabetes, obesity, hypercholesterolemia, hypertension), the heart ageotype was the most significantly associated with heart diseases (atrial fibrillation, heart attack), the muscle ageotype was the most significantly associated with gait impairment, the brain ageotype was the most significantly associated with cerebrovascular disease, and the organismal ageotype was the most significantly associated with Alzheimer's disease. At the whole population level, the relationships between organ age gaps and disease showed the same trends as ageotypes, but more diseases were significantly associated with age gaps due to higher statistical power (65/117, 56%, statistically significant after multiple test correction, FIG. 18e, Table 9).

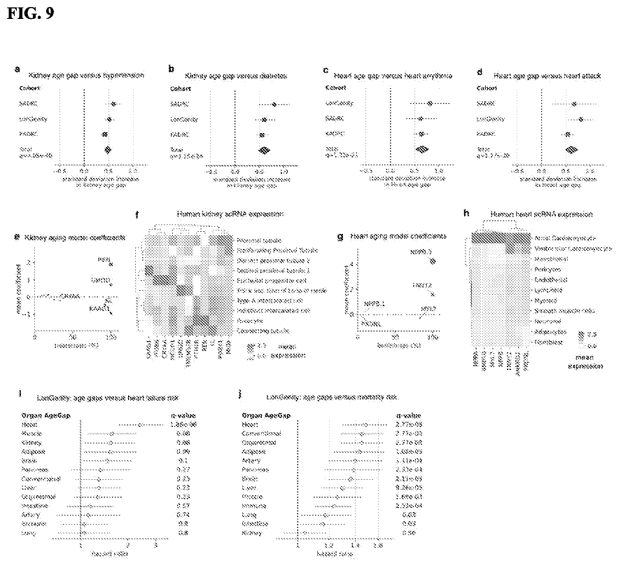

At the population level, the two most significant associations between disease and age gap were between the kidney age gap and metabolic disease traits. Individuals with hypertension had kidneys that were approximately 1 year older than their same-aged peers, while individuals with diabetes had kidneys approximately 1.3 years older (FIG. 9a-b, Tables 7, 9). The third and fourth top associations were between the heart age gap and the heart aging traits atrial fibrillation (2.8 years older) and heart attack (2.6 years older) (FIG. 9c-d). We observed that certain diseases, such as heart attack and Alzheimer's disease, were associated with accelerated aging in virtually all organs, while others had impacts on a particular organ or subset of organs (FIG. 8e, Table 9).

Kidney aging proteins were highly expressed by kidney cell types (FIG. 9e-f) and had known roles in kidney biology and disease. Using feature importance plots, the model identified renin (REN), a kidney enzyme known to regulate blood pressure via the renin-angiotensin pathway³⁴, as an important protein in kidney aging. It also identified the longevity factor klotho (KL)³⁵, as well as multiple proteins with unknown functions including uromodulin (UMOD) and kidney associated antigen 1 (KAAG1), as important kidney aging proteins. Interestingly, UMOD mutations are the major cause of autosomal dominant tubulointerstitial kidney disease³⁶.

Heart aging proteins were expressed primarily by cardiomyocytes (FIG. 9g-h) with recognized roles in heart biology and disease. Pro-brain natriuretic peptide (NPPB), a negative regulator of blood pressure that increases in response to heart damage, and troponin T (TNNT2), a heart muscle protein involved in contraction, had the strongest weights in the heart aging model (FIG. 9g). Both are established clinical markers of acute heart failure²², and NPPB has been previously associated with heart attack risk³⁷. Less well-characterized heart proteins include cardiac myosin light chain (MYL7), peroxidasin like (PXDNL), and bone morphogenetic protein 10 (BMP10). MYL7 is expressed by atrial cardiomyocytes and is a target for hypertrophic cardiomyopathy³⁸ indicating that it could be a repurposing target for heart aging more generally.

Given strong associations between heart aging traits and the heart age gap, we used longitudinal follow-up among healthy participants in the LonGenity cohort to test if organ age was significantly associated with future heart failure risk (FIG. 9i, Table 10). We found that among people with no active disease or clinically abnormal biomarkers at baseline, every 4.1 years of additional heart age (1 standard deviation) conferred an almost 2.5 fold increased risk of heart failure over a 15 year follow-up (23% increased risk per year of heart aging, FIG. 9i). Age gaps from multiple other tissues, but not the conventional aging model, also trended towards significance.

In testing associations between organ age gaps and all-cause mortality, we observed that age gaps from 10 out of 11 organs, the organismal model, and the conventional model were significantly associated with future risk of all-cause mortality after multiple test correction in the LonGenity cohort over 15 years of follow-up (FIG. 9j, Table 11). A standard deviation increase (approximately 4 years of extra organ aging, Table 7) in heart, adipose, liver, pancreas, brain, lung, immune, or muscle age gap each conferred between 15-50% increased all-cause mortality risk. These hazard ratios are of similar size to methylation-based mortality predictors in independent aging cohorts over similar follow-up times despite the fact that organ aging models of the present invention are trained to predict chronological age instead of mortality directly (DNAm GrimAge HR=1.3, 14 year mortality follow-up³⁹).

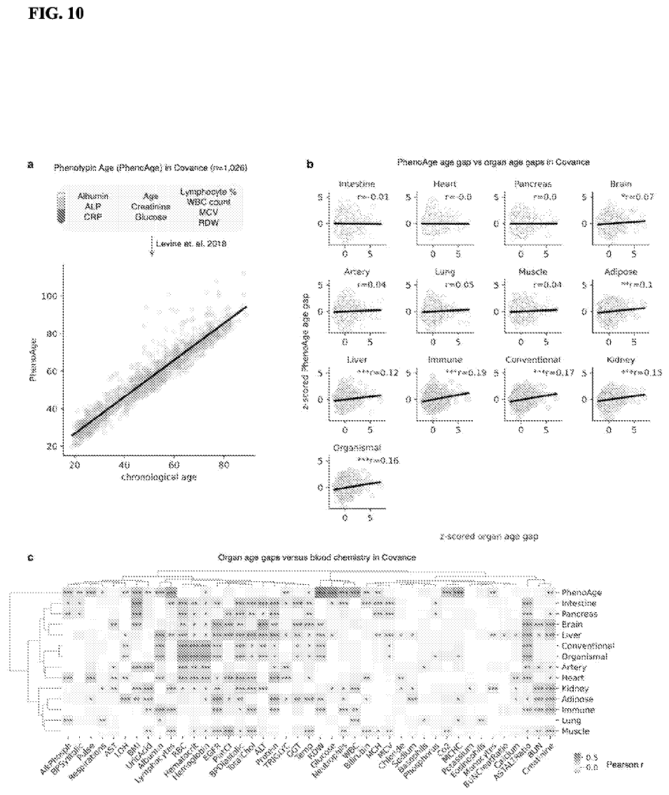

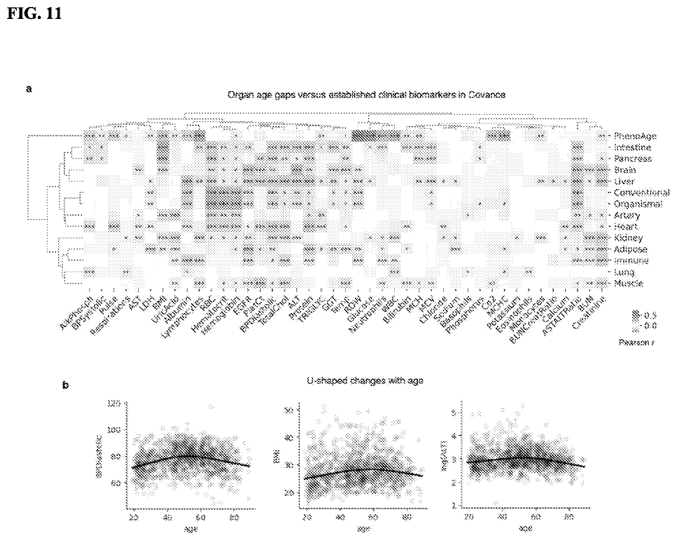

To determine the relationship between organ age and additional markers of health and disease, we tested the associations between organ age gaps and 43 clinical biochemistry and cell count markers in the test cohort Covance (FIG. 10, FIG. 11). We used these markers to calculate Phenotypic age^{12, 19} (PhenoAge), a clinical biochemistry-based aging clock which predicts mortality and morbidity risk for the participants in Covance (FIG. 10a). We found that the PhenoAge age gap was significantly correlated with multiple organ age gaps, but only a small portion of the variance in any model was explained by another (FIG. 10b).

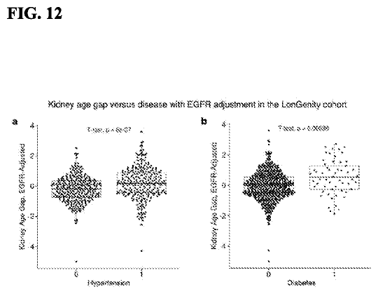

We found 226 out of 559 (40%) associations between organ age gaps and clinical biochemistry markers were significant after multiple testing correction (FIG. 10c, Table 12). The strongest associations included associations between liver age gap and blood AST: ALT ratio, a clinical marker of liver health and function known to change with age (adjusted Pearson r=0.25, q=6.13e-17), and between kidney age gap and serum creatinine, the standard clinical marker of kidney function (adjusted Pearson r=0.23, q=1.65e-16). While these results are highly significant, they only partially explain the relationship between organ age gaps and disease phenotypes. Even after correcting for estimated glomerular filtration rate (EGFR), the kidney age gap is significantly associated with hypertension and diabetes (FIG. 12).

Organ age gap associations with disease and blood biochemistries establish that aging models derived from organ-specific plasma proteins capture disease-relevant heterogeneity of aging within and across individuals that is not captured by other aging clocks or clinical markers. In some embodiments of the present invention, kidney, adipose, brain, immune, and muscle age gaps are significantly positively associated with blood urea nitrogen (BUN), and artery age gap is significantly negatively associated. The strongest association is with the kidney age gap. While blood urea nitrogen (BUN) is non-specific, it is considered a marker of kidney function. In some embodiments, kidney, heart, and artery age gaps are positively significantly associated with aspartate aminotransferase (AST), while brain is significantly negatively associated. Abnormally high AST is may be a sign of liver or heart disease, and moderately high AST may be a sign of elevated cardiovascular risk in middle aged and elderly populations. In some embodiments, brain, control, liver, intestine, kidney, organismal, and pancreas age gaps are significantly negatively associated with alanine transaminase (ALT), while the kidney age gap is significantly positively associated with ALT. Low ALT in the elderly is associated with increased frailty and reduced survival, and has been proposed as a biomarker of aging. (Le Couteur, D. G. et al. The Association of Alanine Transaminase With Aging, Frailty, and Mortality. J. Gerontol. A. Biol. Sci. Med. Sci. 65A, 712-717 (2010).) Abnormally high ALT may be a marker of acute liver damage, although it is also produced by other tissues and is non-specific. In some embodiments, immune, heart, liver, organismal, control, and PhenoAge gaps are significantly negatively associated with albumin levels. The strongest association is with the liver age gap. Albumin is produced by the liver. Lower albumin may be a sign of declining health, and may be low in a diversity of liver, kidney, and digestive diseases as well as in malnutrition/undernutrition. In some embodiments, plasma glucose is significantly positively associated with PhenoAge age gap and kidney age gap, while intestine and liver age gap are significantly negatively associated. The strongest association is with PhenoAge because plasma glucose is the highest weighted input biomarker in the PhenoAge model. Both kidney and intestine age gap are positively associated with diabetes incidence but have distinct associations with plasma glucose. Insulin resistance, glucose response, and glucose levels degrade with age, but insulin levels and glucose response have change more dramatically than fasting blood glucose level. (Bryhni, B., Arnesen, E. & Jenssen, T. G. Associations of age with serum insulin, proinsulin, and the proinsulin-to-insulin ratio: a cross-sectional study. BMC Endocr. Disord. 10, 21 (2010).)

Specific biomarkers of health have a nonlinear relationship to aging outcomes, and in the elderly many relationships between biomarkers and health/mortality/frailty reverse direction compared to young and middle-aged adults. The distribution and mean age of the population that an aging model is trained on will thus impact associations with traits. In some embodiments, diastolic blood pressure has the strongest association with heart aging (adjusted Pearson r=−0.18, q=2.62e-10). Nine organ age gaps (adipose, brain, control, heart, intestine, kidney, liver, muscle, organismal, pancreas) were significantly associated with decrements in diastolic blood pressure, while the opposite association was seen observed the PhenoAge age gap (FIG. 11a, Table 12). Diastolic blood pressure was one of many traits with a U-shaped relationship to aging outcomes (FIG. 11b). Whereas high blood pressure in young and middle-aged adults indicates cardiometabolic dysfunction, in the elderly low blood pressure is common and more strongly associated with mortality and frailty (Protogerou, A. D. et al. Diastolic Blood Pressure and Mortality in the Elderly With Cardiovascular Disease. Hypertension 50, 172-180 (2007), Taylor, J. O. et al. Blood Pressure and Mortality Risk in the Elderly. Am. J. Epidemiol. 134, 489-501 (1991), Boshuizen, H. C., Izaks, G. J., Buuren, S. van & Ligthart, G. J. Blood pressure and mortality in elderly people aged 85 and older: community based study. BMJ 316, 1780-1784 (1998)) though high blood pressure is also detrimental. (Glynn, R. J. et al. Evidence for a positive linear relation between blood pressure and mortality in elderly people. The Lancet 345, 825-829 (1995).) Differences between PhenoAge and the organ age models may arise from differences in the age distribution of the underlying training cohorts for the models. Models of the present invention were trained in the KADRC that comprises a greater proportion of elderly individuals, while PhenoAge was trained in NHANES, with a greater proportion of young individuals. A U-shaped relationship with age and aging outcomes is also observed with BMI (Ng, T. P. et al. Age-dependent relationships between body mass index and mortality: Singapore longitudinal ageing study. PLOS ONE 12, e0180818 (2017). Prospective data in older adults show that while obesity increases mortality and cardiovascular disease risk, the highest risk groups are those with a BMI under 23. The intestine and pancreas age gaps show a negative association with BMI and obesity but a positive association with mortality risk, while the kidney age gap shows a positive association with BMI.

### Example 3—the Role of Brain Aging in Cognitive Decline and Alzheimer's Disease

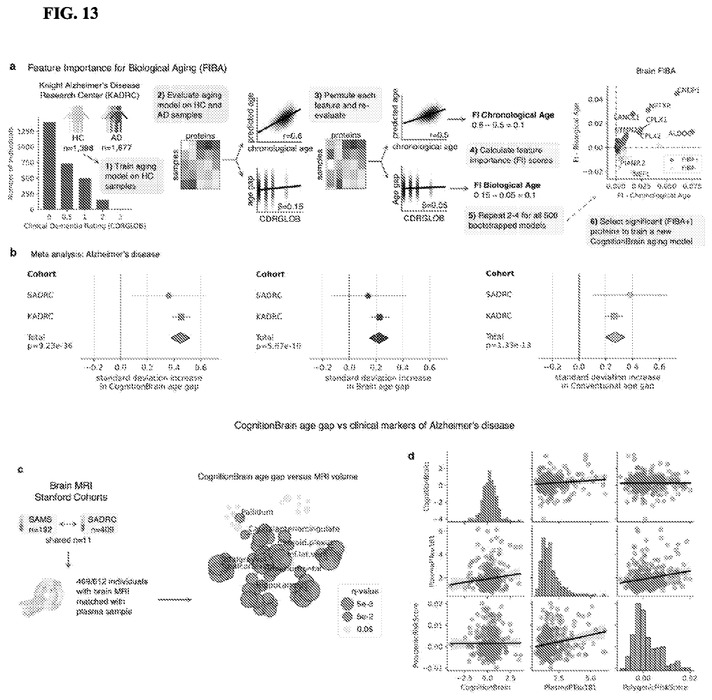

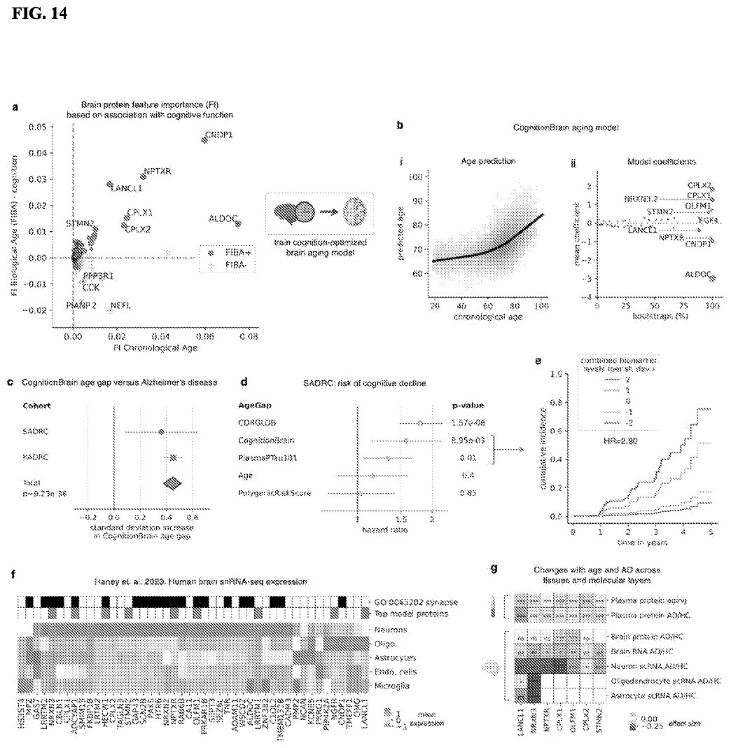

The largest risk factor for neurodegenerative diseases is age. The brain age gap correlated significantly with Alzheimer's disease in held-out participants in the Knight-ADRC, but did not replicate in the Stanford-ADRC (Table 9). To better identify underlying proteins contributed to the brain aging model's predictive abilities for brain aging phenotypes, we developed the Feature Importance for Biological Aging (FIBA) algorithm, which uses feature permutation to generate a per-protein importance score for both chronological and biological age, as defined by a particular age-related trait (FIG. 13a). We applied FIBA to the brain age model using the trait global clinical dementia rating (CDRGLOB) in the Knight-ADRC cohort to understand how brain proteins contribute to the association between the age gap and cognitive decline. We observed that some proteins, such as complexins, increased both the model age prediction accuracy and the age gap association with dementia severity (FIBA+), while others decreased the age gap association with dementia severity (FIBA−) (FIG. 14a).

We used these data to train a second-generation brain aging model termed the CognitionBrain aging model using CDRGLOB FIBA+brain-specific proteins (FIG. 14b, Tables 13-15). This method is similar to the incorporation of biological priors into a LASSO model⁴⁰, and to second-generation methylation aging clocks which are trained jointly on chronological age and aging phenotypes¹². We observed that the CognitionBrain age gap had a stronger association with AD than the first-generation brain age gap and the conventional age gap in the Knight-ADRC cohort (FIG. 13b). This result replicated in the Stanford-ADRC cohort, that was not used to inform feature selection or train the model. In a meta-analysis, individuals with AD had approximately 2 years of additional CognitionBrain aging (meta p=9.23e-36) compared to individuals without AD (FIG. 14c, Tables 15-16). The CognitionBrain age gap was also significantly associated with risk of future dementia progression in both ADRC cohorts. In a meta-analysis, a standard deviation increase in the CognitionBrain age gap conferred a 34% increased risk (meta p=1.30e-15) of a clinically relevant 2-point increase in the Clinical Dementia Rating Sum-of-Boxes score (CDR-SB) within five years (Table 17).

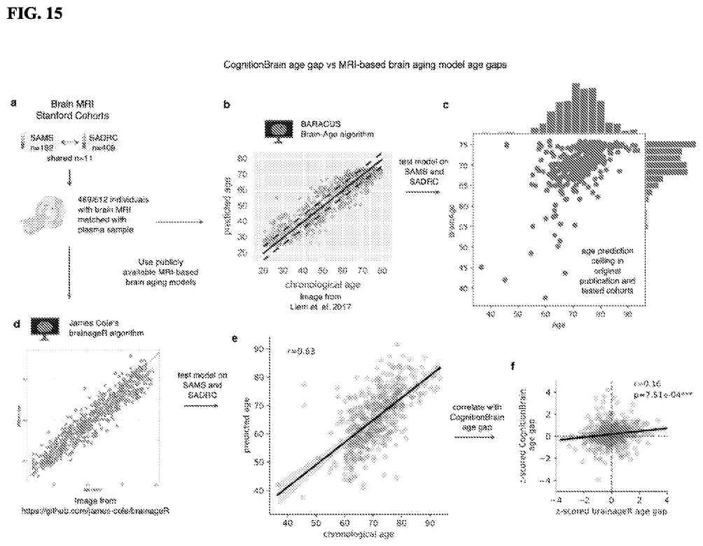

We also tested associations between CognitionBrain age gap and changes in brain volume using plasma matched volumetric MRI in the Stanford-ADRC and SAMS⁴¹ cohorts (FIG. 13c, FIG. 15, and Table 18), and observed that the CognitionBrain age gap significantly predicted brain volume in multiple AD-sensitive regions. We used plasma-matched brain MRI data from 469 individuals in the Stanford-ADRC and SAMS cohorts to assess the relationship between the CognitionBrain age gap and brain region-specific volumes (FIG. 13c, Table 18). 39 out of 65 (60%) associations were significant after multiple hypothesis correction. The most significant associations were negative associations with the superior frontal cortex (adjusted r=−0.20, q=8.49e-5), hippocampus (adjusted r=−0.21, q=1.36e-4), and total cortex (adjusted r=−0.20, q=1.39e-4), whereby individuals with smaller brain region volumes appeared older based on their CognitionBrain age gaps. We observed a negative association with the AD signature region (adjusted r=−0.16, q=3.61e-3), a composite measure of the parahippocampal gyrus, entorhinal cortex, inferior parietal lobes, hippocampus, and precuneus (Walker, K. A. et al. Large-scale plasma proteomic analysis identifies proteins and pathways associated with dementia risk. Nat. Aging 1, 473-489 (2021). We compared plasma proteomics-based brain age of the present invention to 2 MRI brain aging clocks. We used the BARACUS model²¹, a linear support vector machine based aging clock trained on brain MRI-based volumetric data from 1,166 cognitively normal individuals aged 20-80. We also assessed brainageR⁹, a Gaussian Processes based aging clock trained on brain MRI-based volumetric data from n=3,377 cognitively healthy individuals aged 18-92, and which has shown better performance than BARACUS in other studies²¹. The CognitionBrain age gap was positively correlated with the brainageR age gap (r=0.16, p=7.51e-4) (FIG. 13h), but not as strongly as the correlation between CognitionBrain age gap and individual brain volumes (i.e., hippocampus: adjusted r=−0.21, q=1.36e-4), possibly in part because BARaCUS and brainageR do not account for total intracranial volume and may capture more noise.

Given its associations with AD status, cognitive decline risk, and brain volume, we tested whether the CognitionBrain aging model finds use in combination with other biomarkers of Alzheimer's disease and predictors of cognitive decline, including plasma pTau-18142 and an AD polygenic risk score⁴³ to improve stratification of AD patients for future clinical outcomes. We tested a multivariate dementia progression Cox proportional hazard model with baseline CDRGLOB, age, CognitionBrain age gap, plasma pTau-181, and an AD polygenic risk score (FIG. 14d) in the Stanford-ADRC. We observed that the CognitionBrain age gap had the highest adjusted hazard ratio (HR=1.57; p=8.95e-3) of the AD biomarkers, and that both plasma pTau-181 and CognitionBrain age gap were additive for risk prediction (combined HR=2.90, FIG. 14c). Individuals with fluid biomarker levels 2 standard deviations above average had a 75% probability of dementia progression, while individuals with levels 2 standard deviations below average had under a 10% probability of dementia progression within 5 years. Pairwise correlation between the biomarkers also showed that the CognitionBrain age gap was largely independent from other biomarkers (FIG. 15d). Taken together, these data indicate that the CognitionBrain age gap provides clinically relevant molecular information about brain aging not captured by other approaches.

Given the significant associations between the CognitionBrain age model and brain aging metrics, we examined the proteins that the model comprises. Forty seven of the 49 model proteins were detectable in human brain scRNA-sequencing data and mapped to neurons and glia with high specificity (FIG. 14f). Proteins with the largest positive weights in the model (FIG. 3c) included the synaptic proteins complexin 1 (CPLX1), complexin 2 (CPLX2), and neurexin 3 (NRXN3) that have genetic links to cognition and AD,⁴⁴⁻⁴⁸ and stathmin 2 (STMN2) and olfactomedin 1 (OLFM1) that participated in neurite outgrowth and axon growth cone collapse^{49, 50}. Proteins with large negative weights in the model including Aldolase Fructose-Bisphosphate C (ALDOC), neuronal pentraxin receptor (NPTXR), carnosine dipeptidase 1 (CNDP1), and Lanc Like Glutathione S-Transferase 1 (LANCL1). ALDOC, NPTXR, and CNDP1 are expressed in astrocytes, neurons, and oligodendrocytes, respectively and have been proposed as CSF biomarkers for AD^{51, 52}. LANCL1 that is primarily expressed in oligodendrocytes is critical for neuronal health in mouse models^{53, 54}. The model also implicated alterations in the glycosylated extracellular matrix through the proteins tenascin R (TNR), neurocan (NCAN), and heparan sulfate-glucosamine 3-sulfotransferase 4 (HS3ST4) in keeping with the role of the extracellular matrix in brain aging.

We assessed the highest weighted CognitionBrain proteins for their changes with age and AD across plasma in the Knight-ADRC and Stanford-ADRC cohorts, as well as their changes with AD in brain tissue at the protein⁵⁵, bulk RNA⁵⁵, and single-cell RNA levels from publicly available datasets (FIG. 14g). We observed a consistent pattern of decreases in AD brain tissue and increases in the blood with age and AD. These data indicate that the increase of synapse and neurite growth related protein levels in the blood reflect a loss or alteration in protein processing and subsequent shedding of these crucial factors in the brain. A similar inverse relationship between fluid and brain protein levels is seen with amyloid beta, whereby lower CSF AB42 is correlated with increased AB plaques in the brain⁵⁶.

### Example 4—Contributions of Peripheral Organ Aging to Cognitive Decline and AD

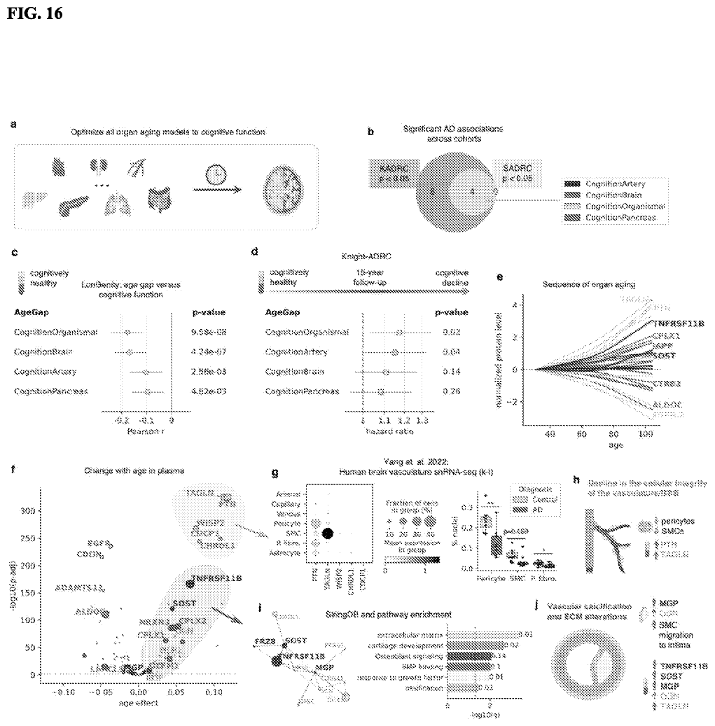

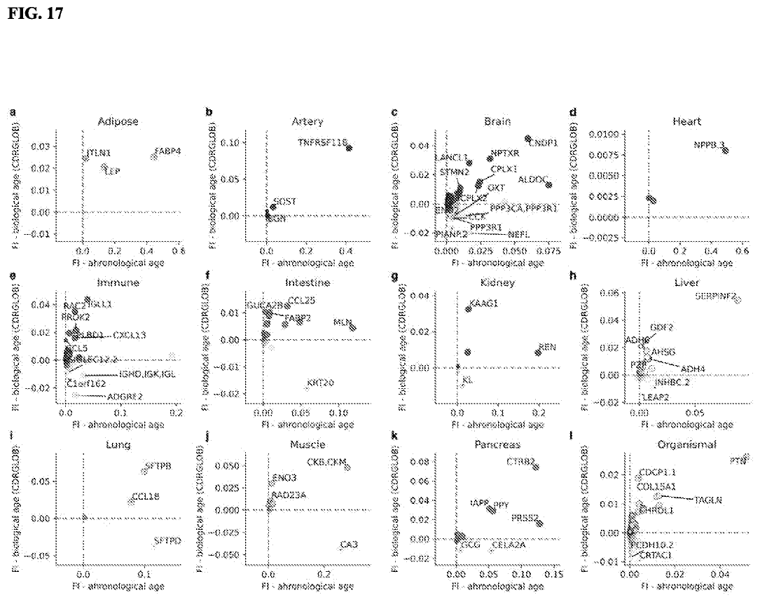

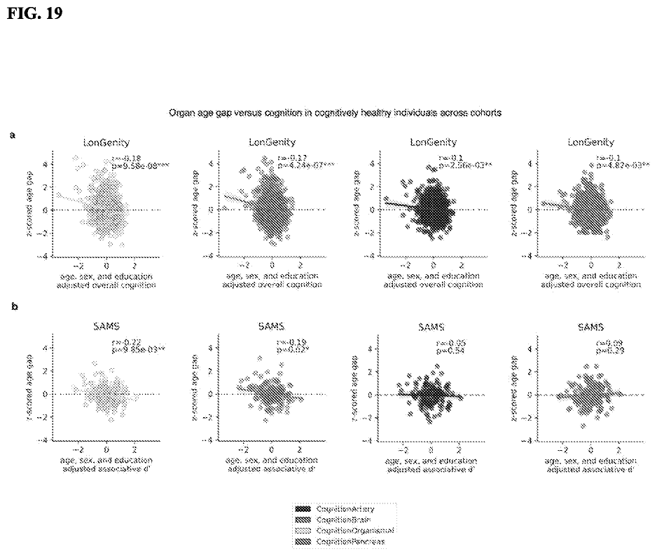

The FIBA optimization framework was applied to other organ aging models to test whether aging of other organs contributes to brain aging phenotypes (FIG. 16a). As with the brain aging model, CDRGLOB FIBA was applied to the aging models using the Knight-ADRC (FIG. 17, FIG. 18). Because the CognitionArtery, CognitionBrain, CognitionOrganismal, and CognitionPancreas age gap associations with AD replicated in both ADRCs (FIG. 16b, FIG. 18c-d) these 4 aging models were selected for further investigation of peripheral vs. central contributions to cognitive decline. To establish the temporal sequence of cognitive decline, we tested if age gaps were associated with cognition in cognitively normal individuals using a composite score of overall cognition in the LonGenity cohort. Decreased cognitive function was significantly associated with the 4 age gaps (FIG. 16c, FIG. 19a). These associations were replicated in the healthy SAMS cohort. Individuals with worse memory recall⁵⁷ had higher CognitionOrganismal and CognitionBrain age gaps (FIG. 19b).

Associations between age gaps and risk of transition from cognitively normal to mild cognitive impairment (MCI) (CDR Global Score 0 to >=0.5) were tested using 15-years of clinical cognitive assessment in the Knight-ADRC (FIG. 16d). We observed that the CognitionOrganismal (HR=1.17, p=0.02) and CognitionArtery (HR=1.15, p=0.04) age gaps significantly predicted conversion to mild cognitive impairment (MCI), with the CognitionBrain (HR=1.11, p=0.14) trending towards significance (FIG. 16d) indicating that changes detected by these aging models occurs early in the causal chain of cognitive decline and neurodegenerative disease.

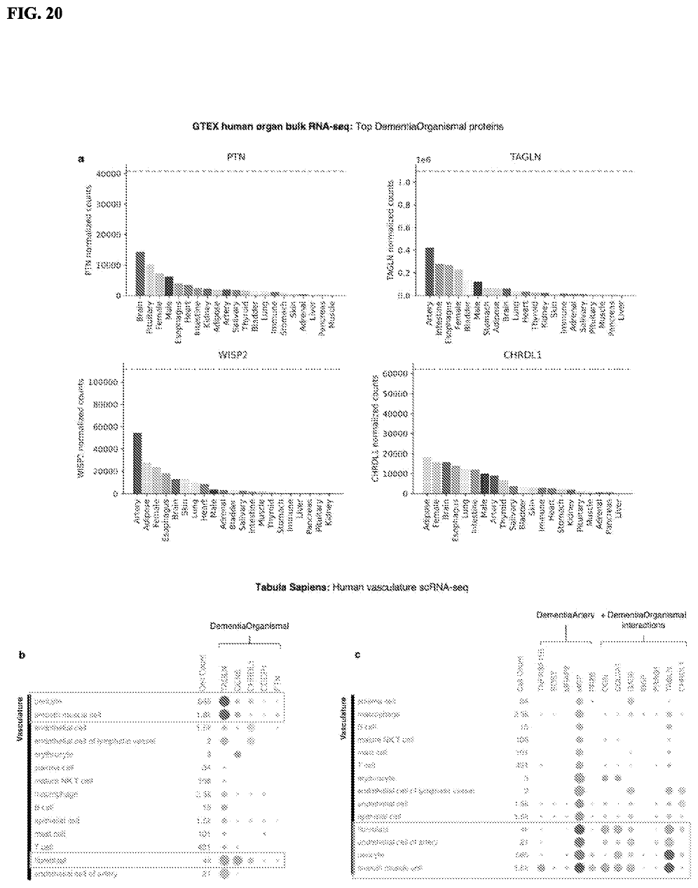

To understand the biological processes and proteins involved in early cognitive decline, we plotted the aging trajectory of the model proteins and found that highly weighted CognitionOrganismal and CognitionArtery proteins changed with age earlier and at a faster rate than CognitionBrain and CognitionPancreas proteins (FIG. 16e). The earliest changes occurred in a highly correlated cluster of CognitionOrganismal proteins: pleiotrophin (PTN), transgelin (TAGLN), WNT1 Inducible Signaling Pathway Protein 2 (WISP2), CUB Domain Containing Protein 1 (CDCP1), and chordin like 1 (CHRDL1; FIG. 16f). Though not organ-specific, these genes were highly expressed in the arteries and brain (FIG. 20a). Single-cell expression of these genes in human vasculature^{58, 59}, indicates that these genes are expressed primarily by smooth muscle cells, pericytes, and fibroblasts (FIG. 16g; FIG. 20b). Loss of brain pericytes, smooth muscle cells, and perivascular fibroblasts is associated with age and AD^{59 61} (FIG. 16g), and pericyte-specific deletion of PTN renders neurons prone to ischemic and excitotoxic injury⁶². The early-changing signature in the CognitionOrganismal model thus corresponds to degenerative changes to the cellular integrity of the brain vasculature, and loss of its neuroprotective functions with aging (FIG. 16h).

The 5 proteins comprising the CognitionArtery model, TNF receptor superfamily member 11b (TNFRSF11B), sclerostin (SOST), melanocortin 2 receptor accessory protein (MRAP2), frizzled related protein (FRZB), and matrix gla protein (MGP) are also primarily expressed in vascular smooth muscle cells, pericytes and fibroblasts⁵⁸ (FIG. 10c) and are implicated in vascular calcification. TNFRSF11B/APOE double knockout mice have increased calcium deposition by vascular smooth muscle cells⁶³, MGP deficiency causing mutations in humans leads to Keutel syndrome, a disease characterized by soft tissue calcification⁶⁴, and SOST and FRZB are negative regulators of WNT signaling that drive calcification and are increased in the plasma of people with vascular calcification^{65, 66}. We observed that CognitionArtery proteins and the vascular signature in the CognitionOrganismal proteins form an interaction network using StringDB (FIG. 16i). Additional model proteins in this interaction network include integrin binding sialoprotein (IBSP), osteoglycin (OGN), collagen type III alpha 1 chain (COL3A1), proline rich and gla domain 1 (PRRG1), and growth arrest specific 6 (GAS6). Together these proteins are enriched in extracellular matrix, cartilage development, and osteoblast signaling pathways and implicate vascular calcification and extracellular matrix alterations as a major component of aging that underlies the early phases of cognitive decline and neurodegenerative disease (FIG. 16i-j).

### Example 5—Machine Learning Protein Quality Control

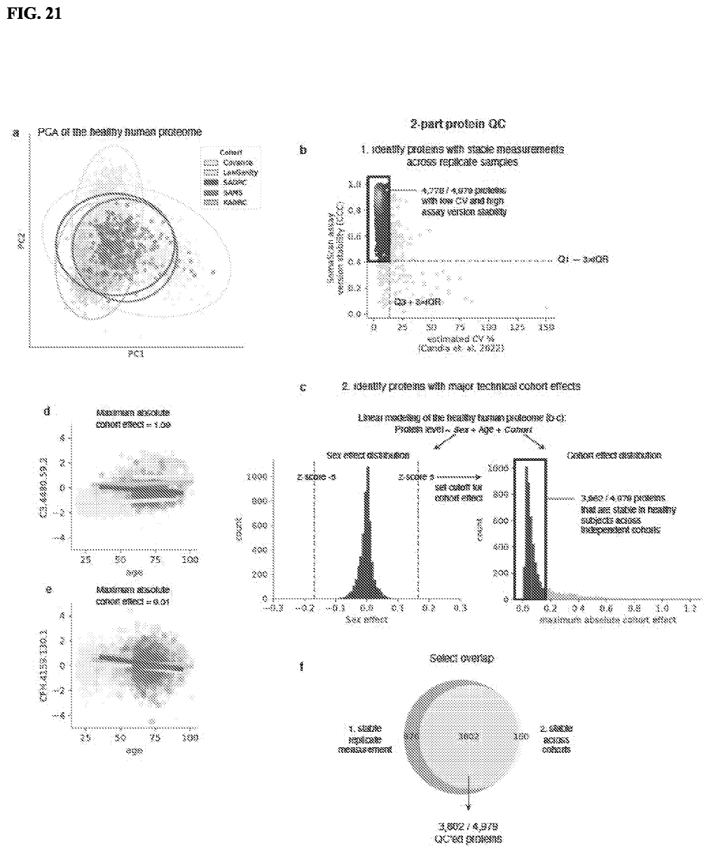

Because healthy individuals across cohorts may differ in principal component analysis (PCA) space (FIG. 21a), we employed a 2-part in silico quality control algorithm to filter to the most replicable protein measurements (FIG. 21b-c). First, we filtered out proteins with poor replicate measurement reproducibility (FIG. 21b) based on Lin's concordance correlation coefficient (CCC) between replicate samples across SomaScan v4 and v4.1 assay versions (data provided by Somalogic) and estimated coefficient of variation (CV) based on replicate samples in Candia, et. al. (Candia, J., Daya, G. N., Tanaka, T. et al. Assessment of variability in the plasma 7k SomaScan proteomics assay. Sci Rep 12, 17147 (2022). Proteins with high outlier values based on 3× the interquartile range for these metrics were deleted. Second, we filtered out proteins that differ wildly between healthy individuals across cohorts independent of age and sex (FIG. 21c). Proteins that differ between healthy individuals across cohorts with an effect size larger than that from biological sex may to be driven by sample handling/technical factors. Specifically, for each protein, a linear model, Protein level˜Sex+Age+Cohort, was tested. Proteins with a cohort effect size that is 5-standard deviations above or below the mean sex effect were filtered out. We used a 5-standard deviation cutoff to include biological signal over removing noise.

### Example 6—Machine Learning Feature Selection for Phenotype-Associated Models

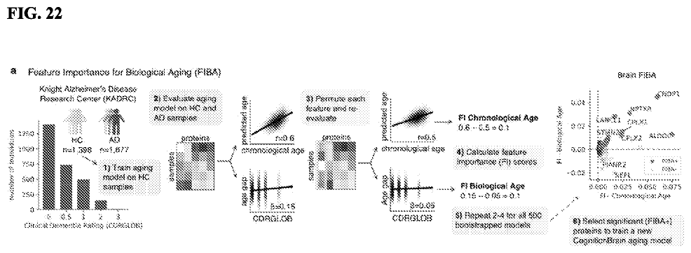

In experiments conducted in development of the present invention, we generated a custom feature importance algorithm identified as Feature Importance for Biological Aging (FIBA) to provide aging models associated with cognitive function (FIG. 22). FIBA is an adaptation of permutation feature importance (PFI) that is conventionally used in machine learning to assess how much a model depends on a given feature for prediction accuracy of the target variable. The PFI score is defined as the decrease in a model's performance when values from a single feature are randomized. For chronological age predictors, the PFI score was calculated as the difference between the model's original prediction accuracy (Pearson correlation between predicted and chronological age) and the model's prediction accuracy after randomization of a single feature. The final PFI score is the mean PFI score from 5 randomizations. FIBA builds on the concept of PFI, and applies it to aging to assess the importance of a feature in measuring biological age, instead of the target variable chronological age. Information about biological age resides in the model age gap and its association with an age-related trait. Thus, randomization of a significant feature reduces the association between the model age gap and the trait in the anticipated direction. The FIBA score for a protein was defined as the difference between the model age gap's original association with a trait and the association with that trait after randomization of a single feature. We applied FIBA to identify aging model protein contributions to associations with cognition using the CDR-Global score. The mean FIBA score after 5 permutations was calculated for the 500 bootstraps for the organ aging models. A protein was defined as significant (FIBA+) if <5% (empirical single-tailed p<0.05) of its FIBA scores across bootstraps was negative. Only proteins with nonzero coefficients in at least 100/500 bootstraps were considered. FIBA+organ-specific proteins were used to train new cognition-optimized aging models from cognitively unimpaired individuals in the Knight-ADRC cohort. In some embodiments, we used least absolute shrinkage and selection operator (LASSO) regression-based aging models. In some embodiments organ aging models were generated from other types of regression approaches including but not limited to ridge regression, elastic net, random forest, XGBoost, and neural networks.

### Example 7—Olink Plasma Proteomic Platform

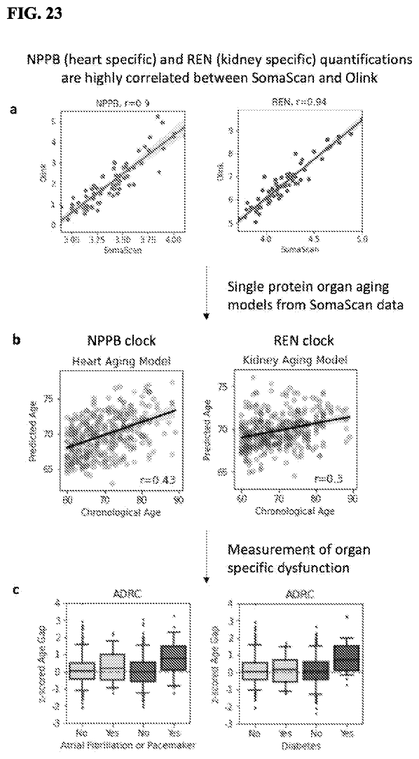

In some embodiments, compositions, methods, systems, kits, and uses of the aging models of the present disclosure are generated from a diversity of -omics platforms. For example, FIG. 23 shows use of the Olink human plasma proteomics platform to derive a heart and kidney aging model associated with heart and kidney disease, respectively. Heart-specific NPPB and kidney specific REN quantifications are highly correlated between Olink and Somascan data.

As provided in Experimental Examples herein, in some embodiments the present invention provides compositions, methods, systems, kits and uses comprising plasma proteomic biomarker panels that predict mortality, organ-specific functional decline, disease risk and progression, and aging heterogeneity between tissues. The biomarker panels are minimally invasive, requiring only a small blood sample, and find utility in measuring the effects of health interventions, such as lifestyle modifications and drug therapies, at the organ level. In some embodiments, the present invention provides an easy-to-use python package termed organage to derive the organ ages of plasma proteomics samples from the SomaScan assay. In some embodiments, the present invention provides molecular measures of aging and disease that improve methylation aging clocks and disease-specific prediction models. In some embodiments, the present invention predicts mortality with effect sizes comparable to models trained specifically to predict mortality and heart disease in independent cohorts^{12, 15, 39, 67, 68}. In some embodiments the present invention adds increased value to conventional biomarkers of Alzheimer's disease, with impacts in other diseases s larger plasma proteomics resources are generated⁶⁹⁻⁷². In some embodiments, the compositions, methods, systems and kits of the present invention comprise additional proteomic coverage, including cell and organ-specific splice isoforms and post-translational modifications together with human gene expression maps at single cell resolution⁷³.

In some embodiments, the compositions, methods, systems and kits of the present invention identify which organ-specific aging proteins are drivers of aging in view of multiple plasma proteins recognized to directly modulate aging phenotypes^{9, 74-76}. Multiple proteins with large weights in biomarker panels, such as KLOTHO, UMOD, MYL7, CPLX1/2^{44, 45} and NRXN3^{46, 47}, have genetic associations with diseases of their respective organs or are validated therapeutic targets, indicating a potential role of these proteins in organ aging. In some embodiments, non-linear machine learning methods such as neural networks and/or random forests improve the accuracy and generalizability of biomarker panels of the present invention in ethnically and geographically diverse populations. In some embodiments, the present invention provides compositions, methods, systems, and kits to non-invasively measure organ health and aging in living people. In some embodiments, organ-specific proteins and the FIBA algorithm provide biomarker panels of physiological age-related proteins that deconvolve different rates of aging within an individual, and measurement of aging at organ-level resolution.

### Experimental Methods

### Human Cohorts

### Covance

Details of the Covance study have been previously published⁷⁷. Covance is a multi-site cross-sectional study of health across the lifespan collected at 5 hospital sites in the United States in 2008. 1028 participants were included in analyses for this study. Cohort demographic characteristics are summarized in Table 15. Exclusion criteria for the study included uncontrolled hypertension, self-reported treatment for a malignancy other than squamous cell or basal cell carcinoma of the skin in the last 2 years, self-reported pregnancy, self-reported chronic infection, autoimmune condition or other inflammatory condition, self-reported chronic kidney or liver disease, chronic heart failure or diagnosed with myocardial infarction in the last 3 months, self-reported diabetes (HbAlc>8% if known), self-reported acute bacterial or viral infection in the past 24 hours or a temperature >38 C within 24 hours of enrollment, self-reported participation in any therapeutic study within 14 days prior of blood sampling, and taking more than 20 mg of prednisone or related drugs. Clinical blood chemistry performed on the same samples, including a complete blood count and comprehensive metabolic panel, lipid panel, and liver function tests. Basic physical data including blood pressure, pulse, and respiratory rate was also collected. Lifestyle information was collected from participants using a survey that asked about smoking, alcohol, exercise, habits, and frequency of consumption of different meats and vegetables.

### LonGenity

Details of the LonGenity cohort have been previously published^{78, 79}. LonGenity is an ongoing longitudinal study initiated in 2008 designed to identify biological factors that contribute to healthy aging. The LonGenity study enrolls older adults of Ashkenazi Jewish descent with age 65-94 years at baseline. Approximately half of the cohort consists of offspring of parents with exceptional longevity, defined as having at least one parent who survived to 95 years of age. The other half of the cohort includes offspring of parents with usual survival, defined as not having a parental history of exceptional longevity. 962 subjects were included in analyses for this study. Cohort characteristics are summarized in Table 15. LonGenity participants are characterized demographically and phenotypically at annual visits that include collection of medical history and physical and detailed neurocognitive assessments. Participants in the LonGenity cohort underwent extensive cognitive examination. The Overall Cognition Composite score was determined by the relative performance of the participant in the Free and Cued Selective Reminding Test, WMS-R Logical Memory I, RBANS Figure Copy, RBANS Figure Recall, WAIS-III Digit Span, WAIS-III Digit Symbol Coding, Phonemic Fluency (FAS), Categorical Fluency, Trail Making Test A, and Trail Making Test B. For each task, a standardized score (z) was calculated based on the population. The z for each task is then combined to create the Overall Cognition Composite.

### Stanford Alzheimer's Disease Research Center (Stanford-ADRC)

Samples were acquired through the National Institute on Aging (NIA)-funded Stanford Alzheimer's Disease Research Center (Stanford-ADRC). The Stanford-ADRC cohort is a longitudinal observational study of clinical dementia subjects and age-sex-matched non-demented subjects. Blood collection and processing were performed according to a standardized protocol to minimize variation associated with blood collection and blood processing. About 10 cc whole blood were collected in a vacutainer EDTA tube (BD Vacutainer EDTA tube) and spun at 3000 RPM for 10 mins to separate plasma, leaving 1 cm of plasma above the buffy coat and taking care not to disturb the buffy coat to circumvent cell contamination. Plasma processing times averaged approximately 1 hour from the time of the blood draw to the time of freezing and storage. Blood draws were done in the morning to minimize the impact of circadian rhythm on protein concentrations. Plasma pTau-181 levels were measured using the Lumipulse G 1200 platform (Fujirebio US, Inc, Malvern, PA) by experimenters blind to diagnostic information as previously described⁴². Healthy control participants were deemed cognitively unimpaired during a clinical consensus conference that included board-certified neurologists and neuropsychologists. Cognitively impaired participants underwent Clinical Dementia Rating and standardized neurological and neuropsychological assessments to determine cognitive and diagnostic status, including procedures of the National Alzheimer's Coordinating Center (naccdata.org/). Cognitive status of impaired participants was determined in a clinical consensus conference that included neurologists and neuropsychologists. Participants were free from acute infectious diseases and in healthy physical condition. 412 participants were included in analyses for this study. Cohort demographics and clinical diagnostic categories are summarized in Table 15.

### Stanford Aging Memory Study (SAMS)

SAMS is an ongoing longitudinal study of healthy aging. Blood collection and processing were performed by the same team and using the same protocol as in Stanford-ADRC. Neurological and neuropsychological assessment were performed by the same team and using the same protocol as in Stanford-ADRC. SAMS participants had CDR=0 and neuropsychological test score within normal range. SAMS participants were deemed cognitively unimpaired during a clinical consensus conference that included neurologists and neuropsychologists. 192 cognitively SAMS participants were studied. 11 were participants in both the Stanford-ADRC and SAMS study.

### Knight Alzheimer's Disease Research Center (Knight-ADRC)

The Knight ADRC (Knight-ADRC) cohort is a National Institute of Aging (NIA) funded longitudinal observational study of clinical dementia subjects and age-matched controls. Participants at the Knight-ADRC undergo longitudinal cognitive, neuropsychologic, imaging, and biomarker assessments including Clinical Dementia Rating (CDR). Among individuals with CSF and plasma data, AD cases correspond to those with a diagnosis of dementia of the Alzheimer's type (DAT) using criteria equivalent to the National Institute of Neurological and Communication Disorders and Stroke-Alzheimer's Disease and Related Disorders Association for probable AD.⁸⁰ AD severity was determined using the Clinical Dementia Rating (CDR®)⁸¹ at the time of lumbar puncture (for CSF samples) or blood draw (for plasma samples). Controls received the same assessment as the cases but were non-demented (CDR=0). Because there are diverse pathologies and disease subtypes in clinically diagnosed individuals, our analysis excluded participants with other neurodegenerative diseases and not AD based on the last clinical and biomarker assessment. CSF and blood for plasma were collected in the morning after an overnight fast, aliquoted, and stored at −80° C. until assayed^{82, 83}. CSF AB and tau levels were measured as described.⁸² 3075 participants were included in the present study.

### Proteomics Data Acquisition and QC

### SomaScan Assay

The SomaLogic SomaScan assay that uses slow off-rate modified DNA aptamers (SOMAmers) to bind target proteins with high specificity was used to quantify the relative concentration of human proteins in plasma. The assay has been used in a diversity of studies^{77, 84}. Two versions of the SomaScan assay were tested in experiments conducted in development of the present invention. The v4 assay (4,979 protein targets) was applied to the Covance and LonGenity cohorts, and the v4.1 assay (7,288 protein targets) was applied to the SAMS, Stanford-ADRC, and Knight-ADRC cohorts. All v4 targets are included in the v4.1 assay based on SeqId, and only the v4 targets were analyzed.

### Somalogic Normalization and QC

Standard Somalogic normalization, calibration, and quality control were performed on the samples^{77, 85-87}. Pooled reference standards and buffer standards are included on each plate to control for batch effects during assay quantification. Samples are normalized within and across plates using median signal intensities in reference standards to control for both within-plate and across-plate technical variation. Samples are further normalized to a pooled reference using an adaptive maximum likelihood procedure. Samples are additionally flagged by SomaLogic if signal intensities deviate significantly from the expected range, and these samples were excluded from analysis. The resulting expression values are the provided data from Somalogic and are considered “raw” data.

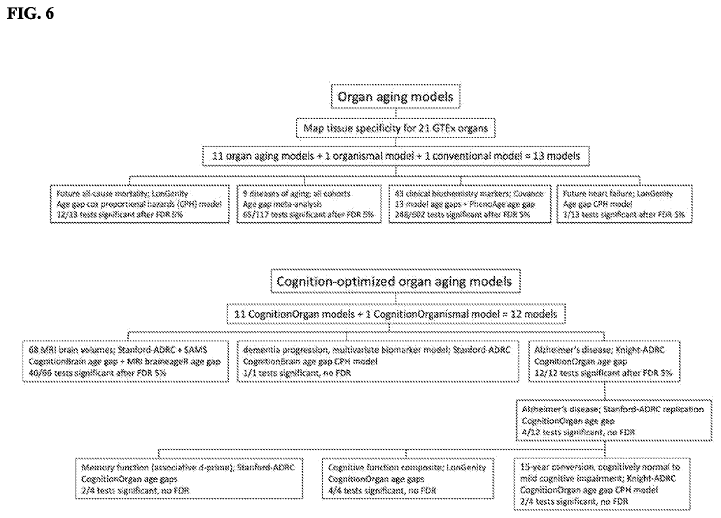

v4→v4.1 multiplication scaling factors provided by Somalogic were applied to the raw v4 assay expression values to allow for direct comparisons across 2 v4 and 3 v4.1 cohorts. Proteins were discarded for which the correlation was low between assay versions v4 and v4.1 and low measured replicate coefficient of variation⁸⁸ (FIG. 6). This resulted in 4,778 proteins for downstream analysis. The raw data were log₁₀ transformed before analysis, as the assay has an expected log-normal distribution.

### Identification of Organ-Enriched Plasma Proteins

The Gene Tissue Expression Atlas (GTEx) human tissue bulk RNA-seq database²⁴ was used to identify organ-enriched genes and plasma proteins (FIG. 2). Tissue gene expression data were normalized using the DESeq2⁸⁹ R package. Organ-enriched genes were defined in accordance with the definition proposed by the Human Protein Atlas²⁵: A gene is enriched if it is expressed at least 4 times higher in a single organ compared to any other organ. Within GTEx, we grouped tissues of the same organ together such that a gene's expression level for a given organ was the maximum gene expression value among its sub-tissues. For example, GTEx brain regions were considered sub-tissues of the brain organ. We define the immune organ, which is not a GTEx tissue, as expression in the blood and the spleen tissues. Organ-enriched genes were mapped to the 4,979 plasma proteins quantified in the v4 SomaScan assay.

### Bootstrap Aggregated LASSO Aging Models

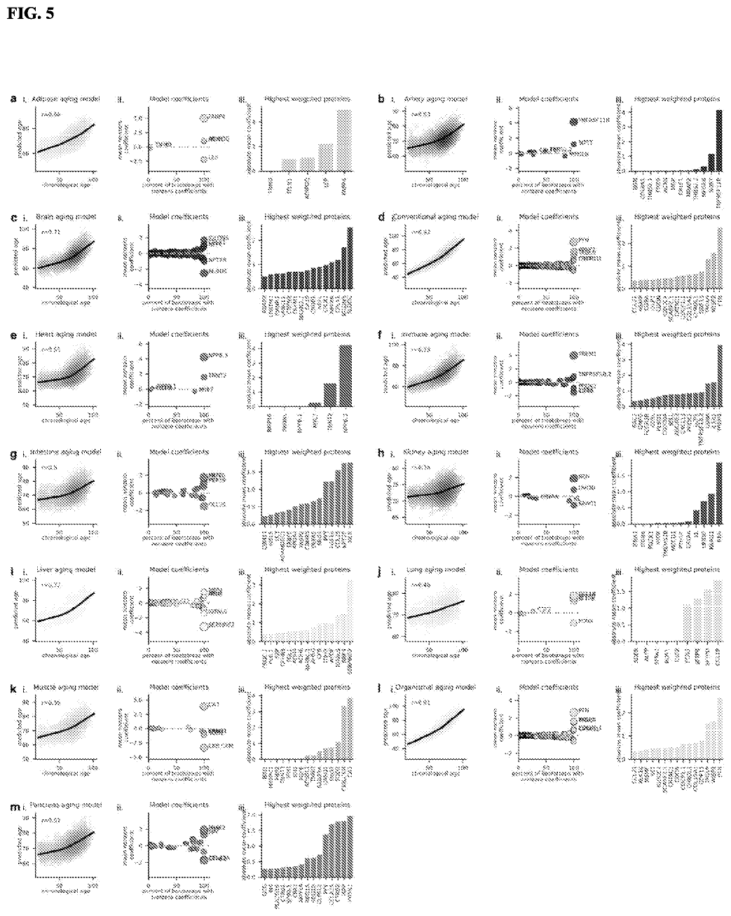

To measure organ biological age using the plasma proteome, we constructed LASSO regression-based chronological age predictors (FIG. 4-5, FIG. 7) using the scikit-learn⁹⁰ python package. Bootstrap aggregation was employed for model training. We resampled with replacement to generate 500 bootstrap samples of training data (Knight-ADRC: 1,398 healthy individuals). Each bootstrap sample was the same size as the training data i.e., 1,398. For each bootstrap sample, we trained a model on z-scored log₁₀ normalized protein expression values with sex (F=1, M=0) as a covariate to predict chronological age. For model training, we performed hyperparameter tuning of the L1 regularization parameter, 2, with 5-fold cross validation using the GridSearchCV function from scikit-learn. To reduce model complexity and avoid overfitting, we selected the highest λ value that retained 95% performance relative to the best model. The mean predicted age from the 500 bootstrap models was used.

We trained models in 1,398 cognitively unimpaired participants from the Knight-ADRC cohort. We evaluated their performance in the Covance (n=1,029), LonGenity (n=962), SAMS (n=192), Stanford-ADRC (n=409) cohorts, and Knight-ADRC cognitively impaired subjects (n=1,677). Models that included sex as a covariate and models trained separately on males and females showed similar age prediction performance on both sexes, so we controlled for sex to extend the generality of the findings and reduce analytic complexity (FIG. 7a-c). A correlation was observed between age measurement accuracy and the number of proteins used as input to each model (FIG. 7c-d). However, specific models with limited protein inputs, such as the adipose model (5 proteins) and heart model (10 proteins) predicted chronological age better than models with more protein inputs (FIG. 5).

### Age Gap Calculation and Independent Validation

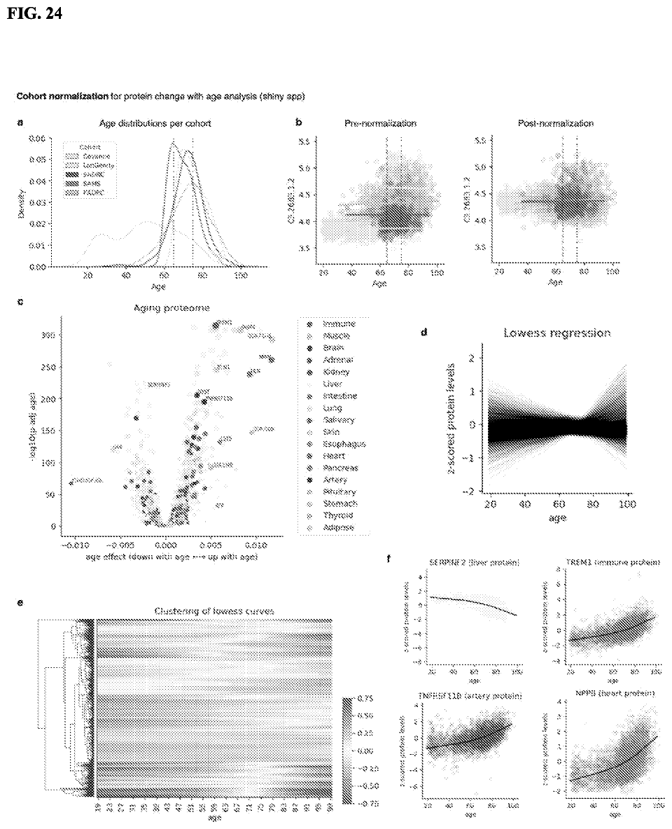

To calculate each individual sample age gap for each aging model, we performed the following steps for each aging model. First, a local regression between predicted and chronological age was fitted using the LOWESS function from the statsmodels⁹¹ python package with fraction parameter set to ⅔ to measure the true population mean (FIG. 7e). A local regression was used in place of a simple linear regression because of evidence that the plasma proteome changes non-linearly with age¹ that is replicated in 5 cohorts (FIG. 24). Individual sample age gaps were then calculated as the difference between predicted age and the LOWESS regression measure of the population mean. Age gaps were calculated separately per cohort to account for cohort differences (FIG. 7e). Age gaps were z-scored per aging model to account for the differences in model variability (FIG. 7f) to allow for direct comparison between organ age gaps in downstream analyses.

### Phenotypic Age Calculation

We used the published coefficients⁹² to calculate the phenotypic age of participants in the Covance cohort using albumin, creatinine, glucose, c-reactive protein, % lymphocyte, mean cell volume, red cell distribution width, alkaline phosphatase, white blood cell count, and age.

### Statistical Methods to Associate Organ Age Gaps with Age-Related Phenotypes

### Study Design

A flowchart of the study design is provided in FIG. 6. Each box in the flowchart was treated as a separate analysis for the purpose of multiple testing correction. Multiple testing correction was done using the Benjamani-Hochberg method, and the significance threshold was a 5% false discovery rate (FDR). To summarize the flowchart, the age gaps from 11 organ aging models, the organismal model, and the conventional model were used in the following analyses: prediction of future mortality in the LonGenity cohort with a Cox proportional hazards model (CPH) (12/13 tests significant after FDR); prediction of future heart disease in the LonGenity cohort with a CPH (12/13 tests significant after FDR); association with 9 diseases of aging in a cross-cohort meta-analysis (66/17 tests significant after FDR); and association with 42 clinical biochemistry markers in the Covance cohort (237/588 tests significant after FDR, PhenoAge gap also tested for 14×42 tests).

The 12 cognition-optimized models (11 organs+organismal model) were tested on additional brain aging phenotypes. The CognitionBrain age gap only was tested for association with 65 MRI brain volumes and an MRI-based brain age gap (40/66 tests significant after FDR). The CognitionBrain age gap only was included in a multivariate CPH model of dementia progression in Alzheimer's disease (1/1 tests significant, no FDR). The 12 cognition-optimized model age gaps were tested for association with Alzheimer's disease status in the Knight-ADRC (12/12 tests significant after FDR), then a replication analysis was performed in Stanford-ADRC (4/12 tests significant at p<0.05, no FDR). The 4 models that replicated, i.e., CognitionBrain, CognitionOrganismal, CognitionArtery, and CognitionPancreas, were then tested for associations with overall cognition in healthy elderly people (LonGenity, 4/4 tests significant, no FDR), memory function in the Stanford-ADRC (2/4 tests significant, no FDR), and 15-year prediction of conversion from normal cognition to mild cognitive impairment in the Knight-ADRC with a CPH model (2/4 tests significant, no FDR).

### Linear Modeling

Estimation of chronological age is insufficient to determine whether an organ aging model measures age-related physiological dysfunction of an organ. To determine whether measured organ age comprises physiologically relevant information, we associated organ age gaps with diverse age-related phenotypes across the Covance, LonGenity, SAMS, Stanford-ADRC, and Knight-ADRC cohorts. Most organ age gap vs trait associations (FIGS. 9a-d; FIG. 14c; FIGS. 8d-e, FIG. 10c, FIGS. 13b-c, FIG. 17, FIGS. 18c-d, FIG. 19) were assessed using linear models controlled for age and sex as follows: age gap˜trait+age+sex and adjusted for multiple testing burden using the Benjamini-Hochberg method when appropriate.

### Meta-Analyses

Meta analyses to compare and aggregate effect sizes and confidence intervals from multiple cohorts were performed in R using the metafor⁹³ package with an inverse variance weighted fixed effects model.

### Cox-Proportional Hazard Modeling

Cox proportional hazards models were used to assess the association between organ age gaps and future risk of mortality, congestive heart failure, and increments in clinical dementia rating using the following model: event risk˜organ age gap+age+sex. Models were tested using the lifelines⁹⁴ python package. Kaplan Meyer curves were generated at population-average covariate values in the relevant subject populations.

### Extreme Agers

Extreme agers were defined as individuals with an age gap value 2-standard deviations above or below the mean (z-scored age gap >2 or z-scored age gap <−2) for at least one aging model. 23% of the population across the cohorts were extreme agers. Extreme agers showed accelerated aging. No individuals displayed extreme youth signatures without extreme aging signature in a different organ (FIG. 8a). To identify different groups of extreme agers with similar aging profiles, we performed k-means clustering (n=13) of the extreme agers. Z-scored age gap values above 2 or below −2 were set to zero before clustering. The clusters showed distinct organ agers (FIG. 1e, FIG. 8b). A multi-organ ager cluster was also identified. Individuals who were extreme agers in at least 5 different organs were manually set to multi-organ agers. Extreme ageotypes (clusters) were associated with major age-related diseases using logistic regression (trait˜e-ageotype) in a cross-cohort meta analysis (FIG. 8d, Table 8)

### Feature Importance for Biological Aging (FIBA)

FIBA is an adaptation of permutation feature importance (PFI)⁹⁵ (FIG. 13a). PFI is conventionally used in machine learning to assess how much a model depends on a given feature for prediction accuracy of the target variable. The PFI score is defined as the decrease in a model's performance when values from a single feature are randomized. For chronological age predictors, PFI score is calculated as the difference between the model's original prediction accuracy (Pearson correlation between predicted and chronological age) and the model's prediction accuracy after randomization of a single feature. The final PFI score is the mean PFI score from 5 randomizations. FIBA builds on the concept of PFI and applies it to the field of aging to assess the importance of a feature in measuring biological age, instead of a target variable chronological age under the assumption that information about biological age resides in the model age gap and its association with an age-related trait. Thus, randomization of an important feature reduces the association between the model age gap and the trait in the expected direction. The FIBA score for a protein is calculated is defined as the difference between the model age gap's original association with a trait and the association with that trait after randomization of a single feature.

We applied FIBA to test aging model protein contributions to associations with cognition using the CDR-Global score. The mean FIBA score after 5 permutations was calculated for the 500 bootstraps for the organ aging models. A protein was defined as significant (FIBA+) if <5% (empirical single-tailed p<0.05) of its FIBA scores across bootstraps was negative. Only proteins with nonzero coefficients in at least 100/500 bootstraps were considered. FIBA+organ-specific proteins were used to train new cognition-optimized aging models from cognitively unimpaired individuals in the Knight-ADRC cohort.

### Biological Pathway Enrichment and Protein-Protein-Interaction (PPI) Analysis

Biological pathway enrichment analyses were performed using g: Profiler⁹⁶ with the human genes set as the background distribution. PPI networks were generated using the STRING database⁹⁷.

### Single-Cell RNA Sequencing Analysis

Pre-processed human heart⁹⁸ and kidney⁹⁹ scRNA-seq data were accessed from studies in the Human Cell Atlas. Pre-processed brain scRNA-seq data were accessed from Michael Haney, Stanford University. Pre-processed human brain vasculature scRNA-seq data were accessed from Yang et. al. 202259. Pre-processed human vasculature scRNA-seq data were accessed from Tabula Sapiens⁵⁸. Gene expression counts data were log (CPM+1) transformed and z-scored for visualization.

### Brain MRI Data from Stanford-ADRC and SAMS Cohorts

### MRI Acquisition

Whole-brain MRI scans were collected from participants in the Stanford-ADRC and SAMS cohorts. MRI data were collected at the Stanford Richard M. Lucas Center for Imaging. 271 participants underwent MRI scanning on a 3T MRI scanner (GE Discovery MR750). T1-weighted SPGR scans were collected (TR/TE/TI=8.2/3.2/900 ms, flip angle=9, 1×1×1 mm) and used to define gray matter volumes. 134 subjects underwent MRI scanning on hybrid PET/MRI scanner (Signa 3 tesla, GE Healthcare). T1-weighted SPGR scan were collected (TR/TE/TI=7.7/3.1/400 ms, flip angle=11, 1.2×1.1×1.1 mm) and used to define gray matter volumes.

### Structural MRI Processing

Region of interest (ROI) labeling was implemented using the FreeSurfer¹⁰⁰ software package version 7 (http://surfer.nmr.mgh.harvard.edu). Structural images were bias field corrected, intensity normalized, and skull stripped using a watershed algorithm. The images underwent a white matter-based segmentation, grey/white matter and pial surfaces were defined, and topology correction was applied to the reconstructed surfaces. Subcortical and cortical ROIs spanning the entire brain were defined in each subject's native space, using the aparc+aseg atlas in FreeSurfer.

MRI brainageR Algorithm

Using matched brain MRI and plasma proteomic data from n=541 samples in SAMS and Stanford-ADRC, we compared plasma proteomic organ clocks with established brain MRI based-clocks, brainageR²⁰ and BARACUS Brain-Age²¹. We used a pre-trained machine learning algorithm (github.com/james-cole/brainageR) and raw T1-weighted MRI scans to measure brain age. The software uses SPM12 (fil.ion.ucl.ac.uk/spm/software/spm12/) to perform tissue segmentation and normalization of individual scans to Montreal Neurological Institute (MNI) template space. The software relies on a model that uses Gaussian process regression to predict brain age on 3,777 participants from seven publicly available datasets (mean age=40.1, range=18-90 years). It applies the results of this training to predict brain age in new T1-w data using the RNifti (version 1.4.5) and kernlab (version 0.9-32) packages within R version 4.2.

We also used a pre-trained algorithm, BARACUS (github.com/bids-apps/baracus; Liem et al. 201721), to measure brain age from FreeSurfer version 5.3 processed T1-w scans. The vertex-wise cortical thickness and surface area values transformed from subject space to fsaverage4 standard space, along with the subcortical volumetric statistics, were used as inputs to BARACUS's linear support vector machine model. This model was trained on 1,166 participants with no objective cognitive impairment (566 female, mean age=59.1, range=20-80 years). The model returned a “stacked-anatomy” prediction among its results used as the measure of brain age for this method.

### MRI Regions of Interest (ROI) Analysis

The volume of the AD signature region was calculated as the sum of the volumes of the parahippocampal gyrus, entorhinal cortex, inferior parietal lobules, hippocampus and precuneus. ROIs were linearly adjusted for estimated total intracranial volume to account for the differences in human size that is unrelated to cognitive function and neurodegeneration. Associations between organ age gaps and adjusted brain ROIs were tested using a linear model controlled for age and sex. Associations were performed for the ROIs in the aparc+aseg atlas.

### Alzheimer's Disease Polygenic Risk Score (PRS) in the Stanford-ADRC Cohort

Alzheimer's disease polygenic risk scores (PRS) were calculated in the Stanford-ADRC cohort to compare to the CognitionBrain age gap. PRSs were determined from whole-genome sequencing (WGS). The Genome Analysis Toolkit (GATK) workflow Germline short variant discovery was used to map genome sequencing data to the reference genome (GRCh38) and to produce high-confidence variant calls using joint-calling¹⁰¹. Six individuals were excluded from further WGS analysis due to discordance between their reported sex and genetic sex. APOE genotype (ε2/ε3/ε4) was determined using allelic combinations of single nucleotide variants rs7412 and rs429358. The independent loci identified in the largest AD GWAS to date were used to compute AD PRS. The 84 variants and their effect size available from Tables 1 and 2 in Bellenguez et al⁴³ were used, in addition to rs7412 (OR=0.6) and rs429358 (OR=3.7). Plink1.9¹⁰² with the “—score” flag was used to formally compute the PRS, while providing the individual genotypes and the list of variants with their effect size as input. Three individuals with pathogenic mutations PSEN1 or GBA were removed from this analysis.

In some embodiments, the present invention provides compositions, methods, systems, kits and uses comprising plasma proteomic biomarker panels that predict mortality, organ-specific functional decline, disease risk and progression, and aging heterogeneity between tissues. The biomarker panels are minimally invasive, requiring only a small blood sample, and find utility in measuring the effects of health interventions, such as lifestyle modifications and drug therapies, at the organ level. In some embodiments, the present invention provides an easy-to-use python package termed organage to derive the organ ages of plasma proteomics samples from the SomaScan assay. In some embodiments, the present invention provides molecular measures of aging and disease that improve methylation aging clocks and disease-specific prediction models. In some embodiments, the present invention predicts mortality with effect sizes comparable to models trained specifically to predict mortality and heart disease in independent cohorts^{12, 15, 39, 67, 68}. In some embodiments the present invention adds increased value to conventional biomarkers of Alzheimer's disease, with impacts in other diseases s larger plasma proteomics resources are generated⁶⁹⁻⁷². In some embodiments, the compositions, methods, systems and kits of the present invention comprise additional proteomic coverage, including cell and organ-specific splice isoforms and post-translational modifications together with human gene expression maps at single cell resolution⁷³.

In some embodiments, the compositions, methods, systems and kits of the present invention identify which organ-specific aging proteins are drivers of aging in view of multiple plasma proteins recognized to directly modulate aging phenotypes^{9, 74-76}. Many proteins with large weights in biomarker panels, such as KLOTHO, UMOD, MYL7, CPLX1/2^{44, 45} and NRXN3^{46, 47}, have genetic associations with diseases of their respective organs or are validated therapeutic targets, indicating a potential role of these proteins in organ aging. In some embodiments, non-linear machine learning methods such as neural networks and/or random forests improve the accuracy and generalizability of biomarker panels of the present invention in ethnically and geographically diverse populations. In some embodiments, the present invention provides compositions, methods, systems, and kits to non-invasively measure organ health and aging in living people. In some embodiments, organ-specific proteins and the FIBA algorithm provide biomarker panels of physiological age-related proteins that deconvolve different rates of aging within an individual, and measurement of aging at organ-level resolution.

Lengthy table referenced here
US20260009803A1-20260108-T00001
Please refer to the end of the specification for access instructions.

Lengthy table referenced here
US20260009803A1-20260108-T00002
Please refer to the end of the specification for access instructions.

Lengthy table referenced here
US20260009803A1-20260108-T00003
Please refer to the end of the specification for access instructions.

Lengthy table referenced here
US20260009803A1-20260108-T00004
Please refer to the end of the specification for access instructions.

Lengthy table referenced here
US20260009803A1-20260108-T00005
Please refer to the end of the specification for access instructions.

Lengthy table referenced here
US20260009803A1-20260108-T00006
Please refer to the end of the specification for access instructions.

Lengthy table referenced here
US20260009803A1-20260108-T00007
Please refer to the end of the specification for access instructions.

Lengthy table referenced here
US20260009803A1-20260108-T00008
Please refer to the end of the specification for access instructions.

Lengthy table referenced here
US20260009803A1-20260108-T00009
Please refer to the end of the specification for access instructions.

Lengthy table referenced here
US20260009803A1-20260108-T00010
Please refer to the end of the specification for access instructions.

Lengthy table referenced here
US20260009803A1-20260108-T00011
Please refer to the end of the specification for access instructions.

Lengthy table referenced here
US20260009803A1-20260108-T00012
Please refer to the end of the specification for access instructions.

Lengthy table referenced here
US20260009803A1-20260108-T00013
Please refer to the end of the specification for access instructions.

Lengthy table referenced here
US20260009803A1-20260108-T00014
Please refer to the end of the specification for access instructions.

Lengthy table referenced here
US20260009803A1-20260108-T00015
Please refer to the end of the specification for access instructions.

Lengthy table referenced here
US20260009803A1-20260108-T00016
Please refer to the end of the specification for access instructions.

Lengthy table referenced here
US20260009803A1-20260108-T00017
Please refer to the end of the specification for access instructions.

Lengthy table referenced here
US20260009803A1-20260108-T00018
Please refer to the end of the specification for access instructions.

Lengthy table referenced here
US20260009803A1-20260108-T00019
Please refer to the end of the specification for access instructions.

Lengthy table referenced here
US20260009803A1-20260108-T00020
Please refer to the end of the specification for access instructions.

### REFERENCES

1. López-Otín, C., Blasco, M. A., Partridge, L., Serrano, M. & Kroemer, G. The Hallmarks of Aging. Cell 153, 1194.1217 (2013).
2. Schaum, N. et al. Ageing hallmarks exhibit organ-specific temporal signatures. Nature 583, 596-602 (2020).
3. Pálovics, R. et al. Molecular hallmarks of heterochronic parabiosis at single-cell resolution. Nature 603, 309-314 (2022).
4. Almanzar, N. et al. A single-cell transcriptomic atlas characterizes ageing tissues in the mouse. Nature 583, 590-595 (2020).
5. Zahn, J. M. et al. AGEMAP: A Gene Expression Database for Aging in Mice. PLOS Genet. 3, e201 (2007).
6. Hajat, C. & Stein, E. The global burden of multiple chronic conditions: A narrative review. Prev. Med. Rep. 12, 284-293 (2018).
7. Kaeberlein Matt, Rabinovitch Peter S., & Martin George M. Healthy aging: The ultimate preventative medicine. Science 350, 1191-1193 (2015).
8. Eggel, A. & Wyss-Coray, T. A revival of parabiosis in biomedical research. Swiss Med. Wkly. 144, (2014).
9. Conboy, I. M. et al. Rejuvenation of aged progenitor cells by exposure to a young systemie environment. Nature 433, 760-764 (2005).
10. Putin, E. et al. Deep biomarkers of human aging: Application of deep neural networks to biomarker development. Aging 8, 1021-1030 (2016).
11. Horvath, S. DNA methylation age of human tissues and cell types. Genome Biol. 14, 3156 (2013).
12. Levine, M. E. et al. An epigenetic biomarker of aging for lifespan and healthspan. Aging 10, 573-591 (2018).
13. Tanaka, T. et al. Plasma proteomic signature of age in healthy humans. Aging Cell 17, e12799 (2018).
14. Lehallier, B. et al. Undulating changes in human plasma proteome profiles across the lifespan. Nat. Med. 25, 1843-1850 (2019).
15. Belsky, D. W. et al. DunedinPACE, a DNA methylation biomarker of the pace of aging. eLife 11, e73420 (2022).
16. Belsky, D. W. et al. Quantification of biological aging in young adults. Proc. Natl. Acad. Sci. 112, E4104-E4110 (2015).
17. Peters, M. J. et al. The transcriptional landscape of age in human peripheral blood. Nat. Commun. 6, 8570 (2015).
18. Rutledge, J., Oh, H. & Wyss-Coray, T. Measuring biological age using omics data. Nat. Rev. Genet. 1-13 (2022) doi: 10.1038/$41576-022-00511-7.
19. Levine, M. E. Modeling the Rate of Senescence: Can Estimated Biological Age Predict Mortality More Accurately Than Chronological Age?. J. Gerontol. A. Biol, Sci. Med. Sci. 68, 667-674 (2013).
20. Cole, J. H. et al. Brain age predicts mortality. Mol. Psychiatry 23, 1385-1392 (2018).
21. Liem, F. et al. Predicting brain-age from multimodal imaging data captures cognitive Impairment. NeuroImage 148, 179-188 (2017).
22, Shrivastava, A., Haase, T., Zeller, T. & Schulte, C. Biomarkers for Heart Failure Prognosis: Proteins, Genetic Scores and Non-coding RNAs. Front. Cardiovasc. Med. 7, (2020).
23. Kim, W. R., Flamm, S. L., Di Bisceglie, A. M. & Bodenheimer, H. C. Serum activity of alanine aminotransferase (ALT) as an indicator of health and disease. Hepatology 47, 1363-1370 (2008).
24. Consortium, T. Gte. The GTEx Consortium atlas of genetic regulatory effects across human tissues. Science 369, 1318-1330 (2020).
25. Uhlên Mathias et al. Tissue-based map of the human proteome. Science 347, 1260419 (2015).
26. Hannum, G. et al. Genome-wide Methylation Profiles Reveal Quantitative Views of Human Aging Rates. Mol. Cell 49, 359-367 (2013).
27. Horvath, S. & Raj, K. DNA methylation-based biomarkers and the epigenetic clock theory of ageing. Nat. Rev. Genet. 19, 371-384 (2018).
28. Galkin, F. et al. Biohorology and biomarkers of aging: Current state-of-the-art, challenges and opportunities. Ageing Res. Rev. 60, 101050 (2020).
29. Bell, C. G. et al. DNA methylation aging clocks: challenges and recommendations. Genome Biol. 20, 249 (2019).
30. McCrory, C. et al. GrimAge Outperforms Other Epigenetic Clocks in the Prediction of Age-Related Clinical Phenotypes and All-Cause Mortality. J. Gerontol. Ser. A 76, 741-749 (2021).
31. Tanaka, T. et al. Plasma proteomic biomarker signature of age predicts health and life span. eLife 9, e61073 (2020).
32. Lehallier, B. et al. Undulating changes in human plasma proteome profiles across the lifespan. Nat. Med. 25, 1843-1850 (2019).
33. Tanaka, T. et al. Plasma proteomic signature of age in healthy humans. Aging Cell 17, e12799 (2018).
34. Sparks, M. A., Crowley, S. D., Gurley, S. B., Mirotsou, M. & Coffinan, T. M. Classical Renin-Angiotensin System in Kidney Physiology. in Comprehensive Physiology 1201˜1228 (John Wiley & Sons, Ltd, 2014). doi: 10.1002/cphy.c130040.
35, Buchanan, S., Combet, E., Stenvinkel, P. & Shiels, P. G. Klotho, Aging, and the Failing Kidney. Front. Endocrinol. 11, (2020),
36. Devuyst, O. & Pattaro, C. The <em>UMOD</em>Locus: Insights into the Pathogenesis and Prognosis of Kidney Disease. J. Am. Soc. Nephrol. 29, 713 (2018).
37. Ho, J. E. et al. Protein Biomarkers of Cardiovascular Disease and Mortality in the Community. J. Am. Heart Assoc. 7, e008108 (2018).
38. Saberi, S. et al. Mavacamten Favorably Impacts Cardiac Structure in Obstructive Hypertrophic Cardiomyopathy, Circulation 143, 606-608 (2021).
39. Lu, A. T. et al. DNA methylation GrimAge strongly predicts lifespan and healthspan. Aging 11, 303-327 (2019).
40. Culos, A. et al. Integration of mechanistic immunological knowledge into a machine learning pipeline improves predictions. Nat. Mach. Intell. 2, 619-628 (2020).
41. Trelle, A. N. et al. Hippocampal and cortical mechanisms at retrieval explain variability in episodic remembering in older adults. eLife 9, e55335 (2020).
42. Wilson, E. N. et al. Performance of a fully-automated Lumipulse plasma phospho-tau181 assay for Alzheimer's disease. Alzheimers Res. Ther. 14, 172 (2022).
43. Bellenguez, C. et al. New insights into the genetic etiology of Alzheimer's disease and related dementias. Nat. Genet. 54, 412-436 (2022).
44. Yu, L. et al. Cortical Proteins Associated With Cognitive Resilience in Community-Dwelling Older Persons. JAMA Psychiatry 77, 1172-1180 (2020).
45. Begemann, M. et al. Modification of Cognitive Performance in Schizophrenia by Complexin 2 Gene Polymorphisms. Arch. Gen. Psychiatry 67, 879-888 (2010).
46. Martinez-Mir, A. et al. Genetic Study of Neurexin and Neuroligin Genes in Alzheimer's Discase. J. Alzheimers Dis. 35, 403˜412 (2013).
47. Hishimoto, A. et al. Neurexin 3 transmembrane and soluble isoform expression and splicing haplotype are associated with neuron inflammasome and Alzheimer's disease. Alzheimers Res. Ther. 11, 28 (2019).
48. Peterson, D. et al. Variants in PPP3R.1 and MAPT are associated with more rapid functional decline in Alzheimer's disease: The Cache County Dementia Progression Study. Alzheimers Dement. 10, 366-371 (2014).
49. Klim, J. R. et al. ALS-implicated protein TDP-43 sustains levels of STMN2, a mediator of motor neuron growth and repair. Nat. Neurosci. 22, 167-179 (2019).
50. Nakaya, N., Sultana, A., Lee, H.-S. & Tomarev, S. I. Olfactomedin 1 Interacts with the Nogo A Receptor Complex to Regulate Axon Growth *. J. Biol, Chem. 287, 37171-37184 (2012).
51. Yin, G. N., Lee, H. W., Cho, J.-Y. & Suk, K. Neuronal pentraxin receptor in cerebrospinal fluid as a potential biomarker for neurodegenerative diseases. Brain Res. 1265, 158-170 (2009).
52. Bader, J. M. et al. Proteome profiling in cerebrospinal fluid reveals novel biomarkers of Alzheimer's disease. Mol. Syst. Biol. 16, e9356 (2020).
53. Tan, H. et al. LanCL1 promotes motor neuron survival and extends the lifespan of amyotrophic lateral sclerosis mice. Cell Death Differ. 27, 1369-1382 (2020).
54. Huang, C. et al. Developmental and Activity-Dependent Expression of LanCL1 Confers Antioxidant Activity Required for Neuronal Survival. Dev, Cell 30, 479-487 (2014).
55. Johnson, E. C. B. et al. Large-scale deep multi-layer analysis of Alzheimer's disease brain reveals strong proteomic disease-related changes not observed at the RNA level. Nat. Neurosci. 25, 213-225 (2022).
56. Tang, W., Huang, Q., Wang, Y., Wang, Z.-Y. & Yao, Y.-Y. Assessment of CSF Aβ42 as an aid to discriminating Alzheimer's disease from other dementias and mild cognitive impairment: A meta-analysis of 50 studies. J. Neurol. Sci. 345, 26-36 (2014).
57. Alexandra N. Trelle et al. Association of CSF Biomarkers With Hippocampal-Dependent Memory in Preclinical Alzheimer Disease. Neurology 96, e1470 (2021).
58. THE TABULA SAPIENS CONSORTIUM. The Tabula Sapiens: A multiple-organ, single-cell transcriptomic atlas of humans. Science 376, eab14896 (2022).
59. Yang, A. C. et al. A human brain vascular atlas reveals diverse mediators of Alzheimer's risk. Nature 603, 885-892 (2022).
60. Sengillo, J. D. et al. Deficiency in Mural Vascular Cells Coincides with Blood-Brain Barrier Disruption in Alzheimer's Disease. Brain Pathol. 23, 303-310 (2013).
61. Soto, I et al. APOE Stabilization by Exercise Prevents Aging Neurovascular Dysfunction and Complement Induction. PLOS Biol. 13, e1002279 (2015).
62. Nikolakopoulou, A. M. et al. Pericyte loss leads to circulatory failure and pleiotrophin depletion causing neuron loss. Nat. Neurosci. 22, 1089-1098 (2019).
63. Callegari, A., Coons, M. L., Ricks, J. L., Rosenfeld, M. E. & Scatena, M. Increased Calcification in Osteoprotegerin-Deficient Smooth Muscle Cells: Dependence on Receptor Activator of NF-κB Ligand and Interleukin 6. J. Vase. Res. 51, 118-131 (2014).
64. Köhler, S. et al. The Human Phenotype Ontology in 2021. Nucleic Acids Res. 49, D1207-D1217 (2021).
65, Qureshi, A. R. et al. Increased circulating selerostin levels in end-stage renal disease predict biopsy-verified vascular medial calcification and coronary artery calcification. Kidney Int. 88, 1356-1364 (2015).
66. Touw, W. A. et al. Association of Circulating Wat Antagonists With Severe Abdominal Aortic Calcification in Elderly Women. J. Endocr. Soc. 1, 26-38 (2017).
67. Wang, T. J. et al. Prognostic Utility of Novel Biomarkers of Cardiovascular Stress. Circulation 126, 1596-1604 (2012).
68. Ganz, P. et al. Development and Validation of a Protein-Based Risk Score for Cardiovascular Outcomes Among Patients With Stable Coronary Heart Disease. JAMA 315, 2532-2541 (2016).
69. Walker, K. A. et al. Large-scale plasma proteomic analysis identifies proteins and pathways associated with dementia risk. Nat. Aging 1, 473-489 (2021).
70. Ferkingstad, E. et al. Large-scale integration of the plasma proteome with genetics and disease. Nat. Genet. 53, 1712˜1721 (2021).
71. Sethi, A., Raj, A., Wright, K. & Melamud, E. Plasma Proteomic Determinants of Common Causes of Mortality, researchsquare.com/article/rs-2626017/v1 (2023) doi: 10.21203/rs.3.rs-2626017/v1.
72. Pietzner, M. et al. Mapping the proteo-genomic convergence of human diseases. Science 374, eabj1541 (2021).
73. null null et al. The Tabula Sapiens: A multiple-organ, single-cell transcriptomic atlas of humans. Science 376, eab14896.
74. Loffredo, F. S. et al. Growth Differentiation Factor 11 Is a Circulating Factor that Reverses Age-Related Cardiac Hypertrophy. Cell 153, 828-839 (2013).
75. Villeda, S. A. et al. Young blood reverses age-related impairments in cognitive function and synaptic plasticity in mice. Nat. Med. 20, 659-663 (2014).
76. Horowitz, A. M. et al. Blood factors transfer beneficial effects of exercise on neurogenesis and cognition to the aged brain. Science 369, 167-173 (2020).
77. Williams, S. A. et al. Plasma protein patterns as comprehensive indicators of health. Nat. Med. 25, 1851-1857 (2019).
78. Gubbi, S. et al. Effect of Exceptional Parental Longevity and Lifestyle Factors on Prevalence of Cardiovascular Disease in Offspring. Am. J. Cardiol. 120, 2170-2175 (2017).
79, Sathyan, S. et al. Plasma proteomic profile of age, health span, and all-cause mortality in older adults. Aging Cell 19, e13250 (2020).
80. Berg, L. et al. Clinicopathologic Studies in Cognitively Healthy Aging and Alzheimer Disease: Relation of Histologic Markers to Dementia Severity, Age, Sex, and Apolipoprotein E Genotype. Arch. Neurol. 55, 326-335 (1998).
81. Morris, J. C. The Clinical Dementia Rating (CDR): Current version and scoring rules. Neurology 43, 2412-a (1993).
82. Fagan, A. M. et al. Cerebrospinal Fluid tau/β-Amyloid42 Ratio as a Prediction of Cognitive Decline in Nondemented Older Adults. Arch. Neurol. 64, 343-349 (2007).
83. Bateman, R. J. et al. Clinical and Biomarker Changes in Dominantly Inherited Alzheimer's Disease. N. Engl. J. Med. 367, 795-804 (2012).
84. Gold, L. et al. Aptamer-Based Multiplexed Proteomic Technology for Biomarker Discovery, PLOS ONE 5, e15004 (2010),
85. SomaLogic. SOMAscan® v4 Data Standardization and File Specification Technical Note.
86. SomaLogic. SomaScan® v4 Data Standardization. (2020).
87. SomaLogic. Technical Specification: Adaptive Normalization Using Maximum Likelihood.
88. Candia, J., Daya, G. N., Tanaka, T., Ferrucci, L. & Walker, K. A. Assessment of variability in the plasma 7k SomaScan proteomics assay. Sei. Rep. 12, 17147 (2022).
89. Love, M. L., Huber, W. & Anders, S. Moderated estimation of fold change and dispersion for RNA-seq data with DESeq2, Genome Biol. 15, 550 (2014).
90. Pedregosa, F. et al. Scikit-learn: Machine Learning in Python. J. Mach. Learn, Res. 12, 2825-2830 (2011).
91. Seabold, S. & Perktold, J. Statsmodels: Econometric and Statistical Modeling with Python. in Proceedings of the 9th Python in Science Conference (eds. Walt, S. van der & Millman, 1) 92-96 (2010). doi: 10.25080/Majora-92bf1922-011.
92. Levine, M. E. et al. An epigenetic biomarker of aging for lifespan and healthspan. Aging 10, 573-591 (2018).
93. Viechtbauer, W. Conducting Meta-Analyses in R with the metafor Package. J. Stat. Softw. 36, 1-48 (2010).
94. Davidson-Pilon, Cameron. (2022). lifelines, survival analysis in Python (v0.27.0). Zenodo. doi.org/10.5281/zenodo.6359609.
95. Breiman, L. Random Forests. Mach. Learn. 45, 5-32 (2001).
96. Raudvere, U. et al. g: Profiler: a web server for functional enrichment analysis and conversions of gene lists (2019 update). Nucleic Acids Res. 47, W191-W198 (2019).
97. Szklarczyk, D. et al. The STRING database in 2021: customizable protein-protein networks, and functional characterization of user-uploaded gene/measurement sets. Nucleic Acids Res. 49, D605-D612 (2021).
98. Litviňuková, M. et al. Cells of the adult human heart. Nature 588, 466-472 (2020).
99. Stewart Benjamin J. et al. Spatiotemporal immune zonation of the human kidney. Science 365, 1461-1466 (2019).
100. Fischl, B. FreeSurfer, NeuroImage 62, 774-781 (2012).
101. Poplin, R. et al. Scaling accurate genetic variant discovery to tens of thousands of samples. 201178 Preprint at doi.org/10.1101/201178 (2018).
102. Chang, C. C. et al. Second-generation PLINK: rising to the challenge of larger and richer datasets. GigaScience 4, $13742-015-0047-8 (2015).

All publications and patents mentioned in the above specification are herein incorporated by reference, Various modifications and variations of the described method and system of the disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

LENGTHY TABLES
The patent application contains a lengthy table section. A copy of the table is available in electronic form from the USPTO web site (https://seqdata.uspto.gov/docdetail?docId=US20260009803A1). An electronic copy of the table will also be available from the USPTO upon request and payment of the fee set forth in 37 CFR 1.19(b)(3).

1. A method of identifying accelerated and slowed aging of an organ in a subject, comprising
a) obtaining a sample from said subject;
b) measuring the concentrations of two or more proteins from sald organ in said sample from said subject wherein said concentrations of said two or more proteins provides a biological age of said organ in health and/or disease; and
c) comparing said biological age of said organ to a chronological age of said subject, wherein a gap between said biological age of said organ and said chronological age of said subject identifies accelerated and slowed aging of said organ.

2. The method of claim 1, wherein said accelerated aging provides a biomarker of dysfunction or disease in said organ.

3. The method of claim 1, wherein said organ is a heart organ, a kidney organ, an immune system organ, a vascular system organ, a muscle organ, an intestine organ, adipose tissue, a liver organ, a lung organ, a pancreas organ or a brain organ.

4. The method of claim 1, wherein said sample is a bodily fluid sample, a whole blood sample, a buffy coat sample, a serum sample, a plasma sample, a urine sample, a saliva sample, a sweat sample, a sputum sample, a semen sample, a mucus sample, a lacrimal fluid sample, a lymph fluid sample, an amniotic fluid sample, an interstitial fluid sample, a cerebrospinal fluid sample, a feces sample, a tissue sample, an organ sample, a dried blood spot sample or a biopsy sample.

5. The method of claim 1, wherein said measuring comprises use of a modified oligonucleotide aptamer-based assay.

6. The method of claim 1, wherein said measuring comprises use of a multiplex immune-based assay.

7. The method of claim 1, wherein said comparing comprises use of a machine learning model.

8. The method of claim 7, wherein said machine learning model comprises Feature Importance for Biological Aging (FIBA).

9. The method of claim 1, wherein said comparing comprises predicting LASSO regression-based chronological age and/or estimating LOWESS regression.

10. The method of claim 1, wherein said biological age is age in decades, years, months, weeks, days and/or hours.

11. The method of claim 1, wherein said chronological age is age in decades, years, months, weeks, days and/or hours since birth.

12. The method of claim 1, wherein said chronological age greater than said biological age identifies slowed aging of said organ.

13. The method of claim 1, wherein said biological age greater than said chronological age identifies accelerated aging of said organ.

14. The method of claim 1, wherein said biological age and said chronological age are the same age.

15. The method of claim 1, wherein said accelerated aging comprises at least one pathophysiology in said organ.

16. The method of claim 1, wherein said accelerated aging comprises two or more pathophysiologies in said organ.

17. The method of claim 1, comprising measuring the concentrations of two or more proteins from two or more organs in said sample from said subject wherein said concentrations of said two or more proteins from said two or more organs provides said biological ages of said two or more organs in health and/or disease.

18. The method of claim 17, wherein said two or more organs comprises five or more organs.

19. The method of claim 17, wherein said two or more organs comprises ten or more organs.

20. The method of claim 17, wherein two or more of said biological ages of said two or more organs is greater than said chronological age.

21. The method of claim 17, wherein said accelerated aging comprises two or more pathophysiologies in said two or more organs.

22. The method of claim 1, comprising measuring the concentrations of two or more proteins from one or more cell types in said organ in said sample from said subject wherein said concentrations of said two or more proteins provides a biological age of said one or more cell types in said organ in health and/or disease.

23. The method of claim 1, wherein said accelerated aging of said organ identified by said comparing directs one or more interventions to prevent and/or to reverse said accelerated aging of said organ in said subject.

24. The method of claim 23, wherein said one or more interventions is one or more of a drug intervention, a drug prophylactic intervention, a drug curative intervention, a drug palliative intervention, a vaccine, a nutritional intervention, an educational intervention, a behavioral intervention, an environmental intervention, a surgical intervention, a radiologic-guided intervention, an applied energy intervention, an applied radiation intervention, a health systems intervention and/or a combination thereof.

25. The method of claim 23, comprising:
d) obtaining a sample from said subject after said intervention;
e) measuring the concentrations of two or more proteins from said organ in said sample from said subject wherein said concentrations of said two or more proteins provides a biological age of said organ in health and/or disease; and
comparing said biological age of said organ to a chronological age of said subject, wherein a gap between said biological age of said organ and said chronological age of said subject identifies accelerated and slowed aging of said organ after said intervention.

26. The method of claim 25, wherein said accelerated aging of said organ identified by said comparing directs one or more further interventions to prevent and/or reverse said accelerated aging of said organ in said subject.

27. The method of claim 24, wherein said drug is a novel drug or a repurposed drug.

28. A method of subject health maintenance, comprising:
a) obtaining two or more interval samples from said subject;
b) measuring the concentrations of two or more proteins from one or more organs in said two or more samples from said subject wherein said concentrations of said two or more proteins provides two or more biological ages of one or more organs in health and/or disease;
c) comparing said biological ages of said one or more organs to a chronological age of said subject, wherein one or more gaps between said biological age of said one or more organs and said chronological age of said subject identifies accelerated and slowed aging of said one or more organs; and
wherein said accelerated aging of said organ identified by said comparing directs one or more interventions to prevent and/or to reverse said accelerated aging of said one or more organs in said subject.

29. The method of claim 28, wherein said comparing further comprises assessment of sex, albumin level, creatinine level, serum glucose level, C-reactive protein level, lymphocyte percent, mean red blood cell volume, red cell distribution width, alkaline phosphatase level, white blood cell count and DNA methylation of said subject.

30. The method of claim 28, wherein said measuring comprises use of one or more organ-specific panels comprising two or more proteins.

31. The method of claim 28, further comprising providing said subject with information comprising said one or more gaps and/or said one or more interventions.

32. The methods of claim 31, wherein said information predicts future organ pathophysiology.

33. A method to test an intervention to prevent and/or to reverse accelerated aging of an organ in a subject, comprising;
a) obtaining a sample from said subject;
b) measuring the concentrations of two or more proteins from said organ in said sample from said subject wherein said concentrations of said two or more proteins provides a biological age of said organ in health and/or disease;
c) comparing said biological age of said organ to a chronological age of said subject, wherein a gap between said biological age of said organ and said chronological age of said subject identifies accelerated and slowed aging of said organ;
d) administering said intervention to said subject;
e) obtaining one or more samples from said subject after said intervention;
b) measuring the concentrations of two or more proteins from said organ in said two or more samples from said subject after said intervention wherein said concentrations of said two or more proteins provides a biological age of said organ in health and/or disease; and
c) comparing said biological age of said organ to a chronological age of said subject before and after said intervention wherein a smaller gap between said biological age of said organ and said chronological age of said subject after said intervention identifies said intervention to prevent and/or reverse said accelerated aging of said organ.
